SEQUENCE LISTING (15 sequences)

SEQ ID NO: 1 - DNA, 4347 bp, artificial
  rep2cap1
ctagagtcctgtattagaggtcacgtgagtgttttgcgacattttgcgacaccatgtggtcacgctgggtatttaagcccgagtgagcacgcagggtctccattttgaagcgggaggtttgaacgcgcagccgccatgccggggttttacgagattgtgattaaggtccccagcgaccttgacgggcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctgggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgcgggcatgcttcagatgctgtttccctgcaagacatgcgagagaatgaatcagaatttcaacatttgcttcacgcacgggacgagagactgttcagagtgcttccccggcgtgtcagaatctcaaccggtcgtcagaaagaggacgtatcggaaactctgtgccattcatcatctgctggggcgggctcccgagattgcttgctcggcctgcgatctggtcaacgtggacctggatgactgtgtttctgagcaataaatgacttaaaccaggtatggctgccgatggttatcttccagattggctcgaggacaacctctctgagggcattcgcgagtggtgggacttgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaaacgtccggtagagcagtcgccacaagagccagactcctcctcgggcatcggcaagacaggccagcagcccgctaaaaagagactcaattttggtcagactggcgactcagagtcagtccccgatccacaacctctcggagaacctccagcaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgcacctgggccttgcccacctacaataaccacctctacaagcaaatctccagtgcttcaacgggggccagcaacgacaaccactacttcggctacagcaccccctgggggtattttgatttcaacagattccactgccacttttcaccacgtgactggcagcgactcatcaacaacaattggggattccggcccaagagactcaacttcaaactcttcaacatccaagtcaaggaggtcacgacgaatgatggcgtcacaaccatcgctaataaccttaccagcacggttcaagtcttctcggactcggagtaccagcttccgtacgtcctcggctctgcgcaccagggctgcctccctccgttcccggcggacgtgttcatgattccgcaatacggctacctgacgctcaacaatggcagccaagccgtgggacgttcatccttttactgcctggaatatttcccttctcagatgctgagaacgggcaacaactttaccttcagctacacctttgaggaagtgcctttccacagcagctacgcgcacagccagagcctggaccggctgatgaatcctctcatcgaccaatacctgtattacctgaacagaactcaaaatcagtccggaagtgcccaaaacaaggacttgctgtttagccgtgggtctccagctggcatgtctgttcagcccaaaaactggctacctggaccctgttatcggcagcagcgcgtttctaaaacaaaaacagacaacaacaacagcaattttacctggactggtgcttcaaaatataacctcaatgggcgtgaatccatcatcaaccctggcactgctatggcctcacacaaagacgacgaagacaagttctttcccatgagcggtgtcatgatttttggaaaagagagcgccggagcttcaaacactgcattggacaatgtcatgattacagacgaagaggaaattaaagccactaaccctgtggccaccgaaagatttgggaccgtggcagtcaatttccagagcagcagcacagaccctgcgaccggagatgtgcatgctatgggagcattacctggcatggtgtggcaagatagagacgtgtacctgcagggtcccatttgggccaaaattcctcacacagatggacactttcacccgtctcctcttatgggcggctttggactcaagaacccgcctcctcagatcctcatcaaaaacacgcctgttcctgcgaatcctccggcggagttttcagctacaaagtttgcttcattcatcacccaatactccacaggacaagtgagtgtggaaattgaatgggagctgcagaaagaaaacagcaagcgctggaatcccgaagtgcagtacacatccaattatgcaaaatctgccaacgttgattttactgtggacaacaatggactttatactgagcctcgccccattggcacccgttaccttacccgtcccctgtaattacgtgttaatcaataaaccggttgattcgtttcagttgaactttggtctcctgtccttcttatcttatcggttaccatggttatagcttacacattaactgcttggttgcgctt

SEQ ID NO: 2 - DNA, 4286 bp, artificial
  rep2cap3
ctagagtcctgtattagaggtcacgtgagtgttttgcgacattttgcgacaccatgtggtcacgctgggtatttaagcccgagtgagcacgcagggtctccattttgaagcgggaggtttgaacgcgcagccgccatgccggggttttacgagattgtgattaaggtccccagcgaccttgacgggcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctgggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacaacctttctgaaggcattcgtgagtggtgggctctgaaacctggagtccctcaacccaaagcgaaccaacaacaccaggacaaccgtcggggtcttgtgcttccgggttacaaatacctcggacccggtaacggactcgacaaaggagagccggtcaacgaggcggacgcggcagccctcgaacacgacaaagcttacgaccagcagctcaaggccggtgacaacccgtacctcaagtacaaccacgccgacgccgagtttcaggagcgtcttcaagaagatacgtcttttgggggcaaccttggcagagcagtcttccaggccaaaaagaggatccttgagcctcttggtctggttgaggaagcagctaaaacggctcctggaaagaagggggctgtagatcagtctcctcaggaaccggactcatcatctggtgttggcaaatcgggcaaacagcctgccagaaaaagactaaatttcggtcagactggagactcagagtcagtcccagaccctcaacctctcggagaaccaccagcagcccccacaagtttgggatctaatacaatggcttcaggcggtggcgcaccaatggcagacaataacgagggtgccgatggagtgggtaattcctcaggaaattggcattgcgattcccaatggctgggcgacagagtcatcaccaccagcaccagaacctgggccctgcccacttacaacaaccatctctacaagcaaatctccagccaatcaggagcttcaaacgacaaccactactttggctacagcaccccttgggggtattttgactttaacagattccactgccacttctcaccacgtgactggcagcgactcattaacaacaactggggattccggcccaagaaactcagcttcaagctcttcaacatccaagttagaggggtcacgcagaacgatggcacgacgactattgccaataaccttaccagcacggttcaagtgtttacggactcggagtatcagctcccgtacgtgctcgggtcggcgcaccaaggctgtctcccgccgtttccagcggacgtcttcatggtccctcagtatggatacctcaccctgaacaacggaagtcaagcggtgggacgctcatccttttactgcctggagtacttcccttcgcagatgctaaggactggaaataacttccaattcagctataccttcgaggatgtaccttttcacagcagctacgctcacagccagagtttggatcgcttgatgaatcctcttattgatcagtatctgtactacctgaacagaacgcaaggaacaacctctggaacaaccaaccaatcacggctgctttttagccaggctgggcctcagtctatgtctttgcaggccagaaattggctacctgggccctgctaccggcaacagagactttcaaagactgctaacgacaacaacaacagtaactttccttggacagcggccagcaaatatcatctcaatggccgcgactcgctggtgaatccaggaccagctatggccagtcacaaggacgatgaagaaaaatttttccctatgcacggcaatctaatatttggcaaagaagggacaacggcaagtaacgcagaattagataatgtaatgattacggatgaagaagagattcgtaccaccaatcctgtggcaacagagcagtatggaactgtggcaaataacttgcagagctcaaatacagctcccacgactggaactgtcaatcatcagggggccttacctggcatggtgtggcaagatcgtgacgtgtaccttcaaggacctatctgggcaaagattcctcacacggatggacactttcatccttctcctctgatgggaggctttggactgaaacatccgcctcctcaaatcatgatcaaaaatactccggtaccggcaaatcctccgacgactttcagcccggccaagtttgcttcatttatcactcagtactccactggacaggtcagcgtggaaattgagtgggagctacagaaagaaaacagcaaacgttggaatccagagattcagtacacttccaactacaacaagtctgttaatgtggactttactgtagacactaatggtgtttatagtgaacctcgccctattggaacccggtatctcacacgaaacttgtgaatcctggttaatcaataaaccgtttaattcgttcagttgaactttggctcttgtgcat

SEQ ID NO: 3 - DNA, 4536 bp, artificial
  rep2cap4
ctagagtcctgtattagaggtcacgtgagtgttttgcgacattttgcgacaccatgtggtcacgctgggtatttaagcccgagtgagcacgcagggtctccattttgaagcgggaggtttgaacgcgcagccgccatgccggggttttacgagattgtgattaaggtccccagcgaccttgacgggcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctgggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggtatgaatctgatgctttttccctgccggcaatgcgagagaatgaatcagaatgtggacatttgcttcacgcacggggtcatggactgtgccgagtgcttccccgtgtcagaatctcaacccgtgtctgtcgtcagaaagcggacgtatcagaaactgtgtccgattcatcacatcatggggagggcgcccgaggtggcctgctcggcctgcgaactggccaatgtggacttggatgactgtgacatggaacaataaatgactcaaaccagatatgactgacggttaccttccagattggctagaggacaacctctctgaaggcgttcgagagtggtgggcgctgcaacctggagcccctaaacccaaggcaaatcaacaacatcaggacaacgctcggggtcttgtgcttccgggttacaaatacctcggacccggcaacggactcgacaagggggaacccgtcaacgcagcggacgcggcagccctcgagcacgacaaggcctacgaccagcagctcaaggccggtgacaacccctacctcaagtacaaccacgccgacgcggagttccagcagcggcttcagggcgacacatcgtttgggggcaacctcggcagagcagtcttccaggccaaaaagagggttcttgaacctcttggtctggttgagcaagcgggtgagacggctcctggaaagaagagaccgttgattgaatccccccagcagcccgactcctccacgggtatcggcaaaaaaggcaagcagccggctaaaaagaagctcgttttcgaagacgaaactggagcaggcgacggaccccctgagggatcaacttccggagccatgtctgatgacagtgagatgcgtgcagcagctggcggagctgcagtcgagggcggacaaggtgccgatggagtgggtaatgcctcgggtgattggcattgcgattccacctggtctgagggccacgtcacgaccaccagcaccagaacctgggtcttgcccacctacaacaaccacctctacaagcgactcggagagagcctgcagtccaacacctacaacggattctccaccccctggggatactttgacttcaaccgcttccactgccacttctcaccacgtgactggcagcgactcatcaacaacaactggggcatgcgacccaaagccatgcgggtcaaaatcttcaacatccaggtcaaggaggtcacgacgtcgaacggcgagacaacggtggctaataaccttaccagcacggttcagatctttgcggactcgtcgtacgaactgccgtacgtgatggatgcgggtcaagagggcagcctgcctccttttcccaacgacgtctttatggtgccccagtacggctactgtggactggtgaccggcaacacttcgcagcaacagactgacagaaatgccttctactgcctggagtactttccttcgcagatgctgcggactggcaacaactttgaaattacgtacagttttgagaaggtgcctttccactcgatgtacgcgcacagccagagcctggaccggctgatgaaccctctcatcgaccagtacctgtggggactgcaatcgaccaccaccggaaccaccctgaatgccgggactgccaccaccaactttaccaagctgcggcctaccaacttttccaactttaaaaagaactggctgcccgggccttcaatcaagcagcagggcttctcaaagactgccaatcaaaactacaagatccctgccaccgggtcagacagtctcatcaaatacgagacgcacagcactctggacggaagatggagtgccctgacccccggacctccaatggccacggctggacctgcggacagcaagttcagcaacagccagctcatctttgcggggcctaaacagaacggcaacacggccaccgtacccgggactctgatcttcacctctgaggaggagctggcagccaccaacgccaccgatacggacatgtggggcaacctacctggcggtgaccagagcaacagcaacctgccgaccgtggacagactgacagccttgggagccgtgcctggaatggtctggcaaaacagagacatttactaccagggtcccatttgggccaagattcctcataccgatggacactttcacccctcaccgctgattggtgggtttgggctgaaacacccgcctcctcaaatttttatcaagaacaccccggtacctgcgaatcctgcaacgaccttcagctctactccggtaaactccttcattactcagtacagcactggccaggtgtcggtgcagattgactgggagatccagaaggagcggtccaaacgctggaaccccgaggtccagtttacctccaactacggacagcaaaactctctgttgtgggctcccgatgcggctgggaaatacactgagcctagggctatcggtacctgcgaatccttcgaccaccttcagtgcggcaaagtttgcttccttcatcacacagtactccacgggacacggtcagcgtggagatcgagtgggagctgcagaaggaaaacagcaaacgctggaatcccgaaattcagtacacttccaactacaacaagtctgttaatcgtggacttaccgtggatactaatggcgtgtattcagagcctcgccccattggcaccagatacctgactcgtaatctgtaattgcttgttaatcaataaaccgtttaattcgtttcagttgaactttggtctctgcgtatttctttcttatctagtttccatgct

SEQ ID NO: 4 - DNA, 4314 bp, artificial
  rep2cap5
ctagagtcctgtattagaggtcacgtgagtgttttgcgacattttgcgacaccatgtggtcacgctgggtatttaagcccgagtgagcacgcagggtctccattttgaagcgggaggtttgaacgcgcagccgccatgccggggttttacgagattgtgattaaggtccccagcgaccttgacgggcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaaaatcaggagagctacctctccttcaactccaccggcaactctcggagccagatcaaggccgcgctcgacaacgcgaccaaaattatgagtctgacaaaaagcgcggtggactacctcgtggggagctccgttcccgaggacatttcaaaaaacagaatctggcaaatttttgagatgaatggctacgacccggcctacgcgggttccatcctctacggctggtgtcagcgctccttcaacaagaggaacaccgtctggctctacggacccgccacgaccggcaagaccaacatcgcggaggccatcgcccacactgtgcccttttacggctgcgtgaactggaccaatgaaaactttccctttaatgactgtgtggacaaaatgctcatttggtgggaggagggaaagatgaccaacaaggtggttgaatccgccaaggccatcctggggggctcaaaggtgcgggtcgatcagaaatgtaaatcctctgttcaaattgattctacccctgtcattgtaacttccaatacaaacatgtgtgtggtggtggatgggaattccacgacctttgaacaccagcagccgctggaggaccgcatgttcaaatttgaactgactaagcggctcccgccagattttggcaagattactaagcaggaagtcaaggacttttttgcttgggcaaaggtcaatcaggtgccggtgactcacgagtttaaagttcccagggaattggcgggaactaaaggggcggagaaatctctaaaacgcccactgggtgacgtcaccaatactagctataaaagtctggagaagcgggccaggctctcatttgttcccgagacgcctcgcagttcagacgtgactgttgatcccgctcctctgcgaccgctcaattggaattcaaggtatgattgcaaatgtgactatcatgctcaatttgacaacatttctaacaaatgtgatgaatgtgaatatttgaatcggggcaaaaatggatgtatctgtcacaatgtaactcactgtcaaatttgtcatgggattcccccctgggaaaaggaaaacttgtcagattttggggattttgacgatgccaataaagaacagtaaataaagcgagtagtcatgtcttttgttgatcaccctccagattggttggaagaagttggtgaaggtcttcgcgagtttttgggccttgaagcgggcccaccgaaaccaaaacccaatcagcagcatcaagatcaagcccgtggtcttgtgctgcctggttataactatctcggacccggaaacggtctcgatcgaggagagcctgtcaacagggcagacgaggtcgcgcgagagcacgacatctcgtacaacgagcagcttgaggcgggagacaacccctacctcaagtacaaccacgcggacgccgagtttcaggagaagctcgccgacgacacatccttcgggggaaacctcggaaaggcagtctttcaggccaagaaaagggttctcgaaccttttggcctggttgaagagggtgctaagacggcccctaccggaaagcggatagacgaccactttccaaaaagaaagaaggctcggaccgaagaggactccaagccttccacctcgtcagacgccgaagctggacccagcggttcccagcagctgcaaatcccagcccaaccagcctcaagtttgggagctgatacaatgtctgcgggaggtggcggcccattgggcgacaataaccaaggtgccgatggagtgggcaatgcctcgggagattggcattgcgattccacgtggatgggggacagagtcgtcaccaagtccacccgaacctgggtgctgcccagctacaacaaccaccagtaccgagagatcaaaagcggctccgtcgacggaagcaacgccaacgcctactttggatacagcaccccctgggggtactttgactttaaccgcttccacagccactggagcccccgagactggcaaagactcatcaacaactactggggcttcagaccccggtccctcagagtcaaaatcttcaacattcaagtcaaagaggtcacggtgcaggactccaccaccaccatcgccaacaacctcacctccaccgtccaagtgtttacggacgacgactaccagctgccctacgtcgtcggcaacgggaccgagggatgcctgccggccttccctccgcaggtctttacgctgccgcagtacggttacgcgacgctgaaccgcgacaacacagaaaatcccaccgagaggagcagcttcttctgcctagagtactttcccagcaagatgctgagaacgggcaacaactttgagtttacctacaactttgaggaggtgcccttccactccagcttcgctcccagtcagaacctcttcaagctggccaacccgctggtggaccagtacttgtaccgcttcgtgagcacaaataacactggcggagtccagttcaacaagaacctggccgggagatacgccaacacctacaaaaactggttcccggggcccatgggccgaacccagggctggaacctgggctccggggtcaaccgcgccagtgtcagcgccttcgccacgaccaataggatggagctcgagggcgcgagttaccaggtgcccccgcagccgaacggcatgaccaacaacctccagggcagcaacacctatgccctggagaacactatgatcttcaacagccagccggcgaacccgggcaccaccgccacgtacctcgagggcaacatgctcatcaccagcgagagcgagacgcagccggtgaaccgcgtggcgtacaacgtcggcgggcagatggccaccaacaaccagagctccaccactgcccccgcgaccggcacgtacaacctccaggaaatcgtgcccggcagcgtgtggatggagagggacgtgtacctccaaggacccatctgggccaagatcccagagacgggggcgcactttcacccctctccggccatgggcggattcggactcaaacacccaccgcccatgatgctcatcaagaacacgcctgtgcccggaaatatcaccagcttctcggacgtgcccgtcagcagcttcatcacccagtacagcaccgggcaggtcaccgtggagatggagtgggagctcaagaaggaaaactccaagaggtggaacccagagatccagtacacaaacaactacaacgacccccagtttgtggactttgccccggacagcaccggggaatacagaaccaccagacctatcggaacccgataccttacccgacccctttaacccattcatgtcgcataccctcaataaaccgtgtattcgtgtcagtaaaatactgcctcttgtggtcattcaatgaataacagcttacaacatctacaaaacctccttgcttgagagtgtggcactctcccccctgtcgcgcgt

SEQ ID NO: 5 - DNA, 4239 bp, artificial
  rep2cap6
ctagagtcctgtattagaggtcacgtgagtgttttgcgacattttgcgacaccatgtggtcacgctgggtatttaagcccgagtgagcacgcagggtctccattttgaagcgggaggtttgaacgcgcagccgccatgccggggttttacgagattgtgattaaggtccccagcgaccttgacgggcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctgggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgcgggcatgcttcagatgctgtttccctgcaaaacatgcgagagaatgaatcagaatttcaacatttgcttcacgcacgggaccagagactgttcagaatgtttccccggcgtgtcagaatctcaaccggtcgtcagaaagaggacgtatcggaaactctgtgccattcatcatctgctggggcgggctcccgagattgcttgctcggcctgcgatctggtcaacgtggatctggatgactgtgtttctgagcaataaatgacttaaaccaggtatggctgccgatggttatcttccagattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggggagcccgtcaacgcggcggatgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagagggttctcgaaccttttggtctggttgaggaaggtgctaagacggctcctggaaagaaacgtccggtagagcagtcgccacaagagccagactcctcctcgggcattggcaagacaggccagcagcccgctaaaaagagactcaattttggtcagactggcgactcagagtcagtccccgacccacaacctctcggagaacctccagcaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgaacatgggccttgcccacctataacaaccacctctacaagcaaatctccagtgcttcaacgggggccagcaacgacaaccactacttcggctacagcaccccctgggggtattttgatttcaacagattccactgccatttctcaccacgtgactggcagcgactcatcaacaacaattggggattccggcccaagagactcaacttcaagctcttcaacatccaagtcaaggaggtcacgacgaatgatggcgtcacgaccatcgctaataaccttaccagcacggttcaagtcttctcggactcggagtaccagttgccgtacgtcctcggctctgcgcaccagggctgcctccctccgttcccggcggacgtgttcatgattccgcagtacggctacctaacgctcaacaatggcagccaggcagtgggacggtcatccttttactgcctggaatatttcccatcgcagatgctgagaacgggcaataactttaccttcagctacaccttcgaggacgtgcctttccacagcagctacgcgcacagccagagcctggaccggctgatgaatcctctcatcgaccagtacctgtattacctgaacagaactcagaatcagtccggaagtgcccaaaacaaggacttgctgtttagccgggggtctccagctggcatgtctgttcagcccaaaaactggctacctggaccctgttaccggcagcagcgcgtttctaaaacaaaaacagacaacaacaacagcaactttacctggactggtgcttcaaaatataaccttaatgggcgtgaatctataatcaaccctggcactgctatggcctcacacaaagacgacaaagacaagttctttcccatgagcggtgtcatgatttttggaaaggagagcgccggagcttcaaacactgcattggacaatgtcatgatcacagacgaagaggaaatcaaagccactaaccccgtggccaccgaaagatttgggactgtggcagtcaatctccagagcagcagcacagaccctgcgaccggagatgtgcatgttatgggagccttacctggaatggtgtggcaagacagagacgtatacctgcagggtcctatttgggccaaaattcctcacacggatggacactttcacccgtctcctctcatgggcggctttggacttaagcacccgcctcctcagatcctcatcaaaaacacgcctgttcctgcgaatcctccggcagagttttcggctacaaagtttgcttcattcatcacccagtattccacaggacaagtgagcgtggagattgaatgggagctgcagaaagaaaacagcaaacgctggaatcccgaagtgcagtatacatctaactatgcaaaatctgccaacgttgatttcactgtggacaacaatggactttatactgagcctcgccccattggcacccgttacctcacccgtcccctgtaattgtgtgt

SEQ ID NO: 6 - DNA, 20 bp, artificial
  upstream primer for cap1 of AAV1
gtctggagcatgactttggc

SEQ ID NO: 7 - DNA, 26 bp, artificial
  downstream primer for cap1 of AAV1
tctagaagcgcaaccaagcagttaat

SEQ ID NO: 8 - DNA, 26 bp, artificial
  upstream primer for cap3 of AAV3
tctagaggtcaaagagactgtgggga

SEQ ID NO: 9 - DNA, 26 bp, artificial
  downstream primer for cap3 of AAV3
tctagatgcacaagagccaaagttca

SEQ ID NO: 10 - DNA, 20 bp, artificial
  upstream primer for cap4 of AAV4
gcggacaggtaccaaaacaa

SEQ ID NO: 11 - DNA, 20 bp, artificial
  downstream primer for cap4 of AAV4
gaaggattcgcaggtaccgg

SEQ ID NO: 12 - DNA, 20 bp, artificial
  upstream primer for cap5 of AAV5
ggatccaggaaaatcaggag

SEQ ID NO: 13 - DNA, 24 bp, artificial
  downstream primer for cap5 of AAV5
tctagacatgaatgggttaaaggg

SEQ ID NO: 14 - DNA, 20 bp, artificial
  upstream primer for cap6 of AAV6
tttgccgacaggtaccaaaa

SEQ ID NO: 15 - DNA, 24 bp, artificial
  downstream primer for cap6 of AAV6
tctagacacacaattacaggggac

(19) United States
(12) Patent Application Publication
(10) Pub. No.: US 2007/0275449 A1
(43) Pub. Date: Nov. 29, 2007

(54) Method for Large-Scale Production, Isolation, Purification and the Uses of Multi-Type Recombinant Adeno-Associated Virus Vectors

(75) Inventors: Xiaobing Wu, Beijing (CN); Hui Cao, Beijing (CN); Xiaoyan Dong, Beijing (CN)

Correspondence Address: SALIWANCHIK LLOYD & SALIWANCHIK;A PROFESSIONAL ASSOCIATION, PO BOX 142950, GAINESVILLE, FL, 32614-2950, US

(73) Assignee: Vector Gene Technology Company Ltd., Beijing (CN)

(21) Appl. No.: 10/576,000

(22) PCT Filed: Oct. 15, 2003
(86) PCT No.: PCT/CN03/00861

§ 371 Date: Jan. 8, 2007

(51) Int. Cl. C12N 7/02; C12N 15/63; C12N 7/01

(52) U.S. Cl. 435/239; 435/235.1; 435/320.1

(57) ABSTRACT

The present invention relates to the method for production, isolation, purification and the uses of recombinant herpes simplex virus vectors loaded with five sero-type adeno-associated virus AAV1, 3, 4, 5 and 6 respectively. The above five sero-type recombinant herpes simplex viruses are HSV-r2c1, HSR-r2c3, HSV-r2c4, HSV-r2c6 respectively. The 1, 3, 4, 5 and 6 sero-type recombinant adeno-associated viruses vectors could be produced by infecting the cells with the recombinant herpes simplex virus vectors.

### TECHNICAL FIELD OF THE INVENTION

The present invention relates to the field of biotechnology. In particular, it relates to methods for large-scale production of multiple serotype recombinant adeno-associated virus vectors (AAV) as well as uses of the recombinant adeno-associated virus vectors such produced.

### BACKGROUND ART OF THE INVENTION

Gene therapy, developed since 1980s, is a brand new model for treating diseases. Different from the traditional drug therapy, it introduces genes per se into human body to correct defective genes or exert therapeutic effects. As compared with traditional therapies, gene therapy is obviously advantageous in that it is administered once, but has a long-term effect, and is much closer to the natural state of human gene expression. Therefore, it is safer and more effective. According to statistics up to 2002, more than 800 clinical solutions for gene therapy have been approved all over the world, which primarily target at diseases that seriously threaten human health, such as genetic diseases, tumor, infectious diseases as well as various metabolic diseases, and more than 3400 people have received such therapy.

The key issue in gene therapy is to find an approach for safe and effective introduction of therapeutic genes into the human body, and as well long-term expression of the therapeutic genes. Methods generally employed are physical and biological methods.

The physical method introduces therapeutic genes into cells by means of calcium phosphate, electrotransformation, liposome, etc. The method is good at security, but not efficient, and thus, generally limited to experimental research.

The biological method uses an organism having a natural capacity of infecting human body, primarily a virus, as a vector, wherein therapeutic gene is assembled into the virus vector by gene recombination technology, and is introduced into human body via virus infection. Due to the high transduction efficiency of virus vectors, most gene therapy solutions adopt such a method now (Morgan, R. A. and W. F. Anderson, Annu. Rev. Biochem. 62:191-217, 1993).

Virus Vector System

At present, the common available virus vectors are retroviral vector, adenoviral vector, adeno-associated virus vector, herpes simplex virus vector, etc.

Retroviral vector was the most frequently used ones once, since it is capable of inserting therapeutic genes into chromosomes of human cells and stably dividing with cell division, thus, stabilizing the therapeutic gene for lasting expressions. However, as the insertion is random, there is a hidden danger of damaging the function of human normal gene.

Adenoviral vector genes do not insert into human chromosomes, and also have more infectious cell varieties than retrovirus. However, since therapeutic genes are not inserted into human chromosomes, there is a need for several repeated administration; unfortunately, as adenovirus has strong immunogenicity, the repeated use in human body elicits neutralizing antibody, and lower the therapeutic effect. Thus the using times of adenoviral vectors on patients are limited.

Herpes simplex virus vector is characterized by a high transduction rate, the ability to infect both dividing and non-dividing cells, and the ability to conduct in the reverse direction of axonal conduction in nerve system, which promise better applications in nervous system. At present, its use is limited primarily due to its neurotoxicity. Thus, the virus vector remains in the research stage.

AAV Vector

The application of adeno-associated virus (AAV) as a vector for gene therapy has been rapidly developed in recent years. Wild-type AAV could infect, with a comparatively high titer, cells not only in division phrase but also in non-division phrase, or tissues of mammal, including human, and also can integrate into in human cells at specific site (on the long arm of chromosome 19) (Kotin, R. M., et al., Proc. Natl. Acad. Sci. USA 87: 2211-2215, 1990) (Samulski, R. J., et al., EMBO J. 10: 3941-3950, 1991). AAV vector without rep and cap genes loses the specificity of site-specific integration, but could still mediate long-term stable expression of exogenous genes. AAV vector exists in cells in two forms, wherein one is episome outside of the chromosome; another is integrated into the chromosome, with the former as the major form. Moreover, AAV has not been hitherto found to be associated with any human disease, nor any change of biological characteristics arising from the integration has been observed. Thus, it has an obviously better security than retroviral vector and adenoviral vector, which are related to human cancer and respiratory diseases, respectively.

AAV vector has been greatly developed in recent years for its low immunogenicity, capacity of expressing exogenous genes in a long term with stability and infecting various types of histiocytes, etc. However, AAV vector has yet to be improved.

Currently used AAV vectors are mainly of serotype 2, i.e. AAV2, which has been studied for nearly 30 years. Experiments show that recombinant AAV2 (rAAV2) are good gene transfer vectors in a variety of tissues (Monahan, P and R. Samulski, 2000, Gene Ther.7: 24-30). However, with the increase of in vivo experiments, the limitations of rAAV2 vectors become more and more obvious (Bartlett, J. S., R. Wilcher, and R. J. Amulski. 2000. J. Virol. 74: 2777-2785. ) (Davidson, B., C. Stein, J. Heth, et al. 2000. Proc. Natl. Acad. Sci. USA 97: 3428-3432. ): the infection efficiency of AVV2 is high in some tissues, but quite low in others. In addition, the human body could produce neutralizing antibody against AAV infection, and about 85% of normal population develops the antibody against AAV2.

To further enhance the infection efficiency and host range of AAV vectors, researchers have attempted a variety of methods, such as target delivery of AAV mediated via bifunctional antibody (Bartlett, J. S., J. Kleinschmidt, R. S. Boucher, and R. J. Samulski. 1999. Nat. Biotechnol. 17:181-186), mutagenesis screening of capsid proteins (Girod, A., M. Ried, C. Wobus, et al. 1999. Nat. Med. 5: 052-1056. ) (Wu, P., W. Xiao, T. Conlon, J. Hughes, et al. 2000. J. Virol. 74: 8635-8647), etc., which have gained some achievements. Recently, researches have focused on the tissue specificity of various serotype AAV vectors, and it is possible to obtain AAV vectors having different transduction efficiencies to various tissues by using the natural infection characteristics of various AAV serotypes. (Chao, H., Y. Liu, J. Rabinowitz, C. Li, R. J. Samulski, et al. 2000. Mol. Ther. 2: 619-623. )(Chiorini, J., L. Yang, Y. Liu, B. Safer, and R. Kotin. 1999. J. Virol. 73:1309-1319. )(Chiorini, J. A., B. Zimmermann, L. Yang, R.et al. 1998. Mol. Cell. Biol. 18: 5921-5929).

AAV and the Genome Structure Thereof

AAV is a small size and no capsid virus, which contains single-stranded DNA with positive strands substantially equal to negative strands in number. AAV belongs to the genus of Parvoviridae, and replication thereof requires the existence of a helper virus (Kotin, RM. 1994. Hum. Gene Ther. 5: 793-801). It is reported in literatures that the major AAVs of Primates are six kinds of serotypes, respectively named AAV1, AAV2, AAV3, AAV4, AAV5 and AAV6 (Baclunann Pa., MD. Hoggan, J L. Melnick, 1975, Parvoviridae, Intervirology 5: 83-92)(Bantel-Schaal U., and H. zur Hausen. 1998. Virology 134: 52-63)(Rutledge. E A., C L. Halbert, and D W. Russell. 1998. J. Virol. 72: 309-319). So far, the whole sequences of AAV1, 2, 3, 4, 5, 6 have been determined, and the homologies of various serotype genomes are between 52 and 82% (Bantel-Schaal U., and H. zur Hausen. J. Virol. 1999, 73: 939-947). In addition, more and more new AAV serotypes are reported, such as AAV7 and AAV8, etc.

Among these AAVs, AAV 2 is the clearest studied one, whose genome is a single-stranded DNA of 4680 bp in length (Laughlin, C. A., J. D. Tratschin, H. Coon, and B. J. Carter. 1983. Gene 23: 65-73). The genome is flanked by inverted terminal repeats (ITR), which form palindromic hairpin structures with breaks to stabilize the single-stranded genome. There are only two large open reading frames (ORF) in the genome, encoding rep and cap genes, respectively (Srivastava A E., Lusby and K I. Bems. 1983. J. Virol. 45: 555-564).

AAV2 genome has rep gene on the left, which encodes the non-structural protein Rep of AAV initiated by promoters p5 and p19, respectively, and coupled with the differential splicing mRNA transcripts resulting in the production of four proteins: Rep78, Rep68, Rep52 and Rep40. Rep proteins act in controlling transcription of AAV, involving in AAV replication, and playing an important role in generating daughter genomes and packaging virion. Rep78 and Rep68 bind specifically to ITR at the terminal resolution site (trs) and GAGY repeat motif, so as to initiate the replicating process of AAV genome from single strand to double strand (Chiorini, J. A., S. M. Wiener, R. M. Kotin, R. A. Owens, and B. Safer. 1994. J. Virol. 68: 7448-7457). The binding of Rep to DNA, and the terminal resolution process are also the process of site-specific insertion of AAV genome into AAVS1 site of the long arm of 19 chromosome (Kotin, R. M., J. C. Menninger, D. C. Ward, and K. I. Berns. 1991. Genomics 10: 831-834). In ITR, the trs and GAGC repeat motif are the center of AAV genome replication. Thus, although ITR sequences in various AAV serotypes are not always the same, they could still form hairpin structures and have Rep binding sties (such as GAGY of AAV2), and trs. 19 bp downstream of rep78, rep68 are additional two rep genes, respectively expressing Rep52 and Rep40, whose promoter is p19. Rep52 and Rep40 cannot bind DNA, but have the ATP dependent DNA helicase activity (Smith, R., and R. M. Kotin. 1998. J. Virol. 72: 4874-4881). The conservation degree of Rep protein in AAV1, 2, 3, 4, 6 is comparatively higher, wherein the homology of Rep78 among the viruses reaches 89-93% (Chiorini J. A., L. Yang, Y. Liu, and RM. Kotin. 1997. J. Virol. 71: 6823-6833. Muramatsu. S I. H. Mizukami, N S. Young and K E. Brown. 1996. Virology 221: 208-217).

The right portion of AAV2 genome is cap gene, which encodes capsid proteins VP1, VP2 and VP3, wherein VP3 has the smallest molecular weight, but the largest quantity, while VP1 has the largest molecular weight, but the smallest quantity. In a mature AAV particle, the ratio of VP1, VP2, VP3 is 1:1: 20. VP1 is indispensable in forming infectious AAV, VP2 assists VP3 to enter into nucleus, and VP3 is the major protein in composing a AAV particle (Muzyczka. N. 1992. Curr. Top. Microbiol. Immunol. 158: 97-129). Different from Rep, Cap protein has a comparatively low conservative degree in various serotypes of AAV, which is the primary cause for different host ranges and specificities in different serotypes of AAV.

Comparison of Homologies of Various Serotypes of AAV

There are six serotypes of Primates AAV as reported in literature, respectively named AAV1, AAV2, AAV3, AAV4, AAV5 and AAV6, wherein only AAV5 is originally isolated from human body (Bantel-Schaal, and H. zur Hausen. 1984. Virology 134: 52-63), while the remaining five serotypes are all found in the study of adenovirus (Ursula Bantel-Schaal, Hajo Delius and Harald zur Hausen. J. Virol. 1999, 73: 939-947). So far, the whole sequences of all the six serotypes of AAV have been identified, (John Chiorini, Frank Kim, Linda Yang, and Robert Kotin. J. Virol. 1999, 73:1309-1319). The homologies of the genomes ofAAV serotypes 1, 2, 3, 4, 6, except AAV5, are generally high, specifically ITR and Rep regions, wherein the homology of Rep in AAV1, 2, 3, 4, 6 is up to 89-93%. Therefore, Rep between AAV serotypes 1, 2, 3, 4, 6 could be used to identify ITR from another serotype, and support the packaging thereof (Chiorini J, L. Yang, Y. Liu, B. Safer, and M.Kotin. 1997. J. Virol. 71: 6823-6833)(Muramatsu, S., H. Mizukami, N. Young, and K. Brown. 1996. Virology 221: 208-217). However, the homology of Rep of AAV5 to those of other serotypes is only 67% (Ursula Bantel-Schaal, Hajo Delius and Harald zur Hausen. J. Virol. 1999, 73: 939-947)(John Chiorini, Frank Kim, Linda Yang, and Robert Kotin. J. Virol. 1999, 73:1309-1319). Therefore, Rep of AAV5 could not identify ITR of other AAV serotypes.

Cell Receptors for AAVs

As compared with Rep, the homology of Cap in various AAV serotypes is comparatively lower. The amino acid homologies of Cap in AAV1, AAV2, AAV3, AAV5, AAV4, AAV6 are in a range of 45˜80%, wherein the homology of AAV1 and AAV6 is the highest (the amino acid homology of Capsid is more than 99%), and the homology of Cap of AAV5 with those of other serotypes are the lowest (Ursula Bantel-Schaal, Hajo Delius and Harald zur Hausen. J. Virol. 1999, 73: 939-947). This serves as the foundation for different host ranges and cell specificities in various serotypes. The host range and cell specificity are determined by the type and number of corresponding receptors in the infected cells. Currently, the receptors of serotypes such as AAV2, AAV3, AAV4, AAV5 are relatively clear in the study. The cell receptor of AAV2, AAV3 serotypes is heparin sulfate proteoglycan, whose receptor binding site is on VP3 protein of AAV2, and whose coreceptor (assisting AAV to enter into the cell) is fibroblast growth factor receptor 1 and integrin αVβ5 (Qing, K., C. Mah, J. Hansen, S. Zhou, V. Dwarki, and A. Srivastava. 1999. Nat. Med. 5: 71-77)(Summerford, C., J. S. Bartlett, and R. J. Samulski. 1999. Nat. Med. 5: 78-82). The cell receptor of AAV4 and AAV5 is sialic acid (Walters R W, Yi S M, Keshavjee S, Brown K E, et al. J Biol. Chem. 2001 276: 20610-6) without heparin sulfate binding site. Thus, the specificity of AAV5 is greatly different from AAV2, etc.; particularly, the infection efficiency of AAV5 is much higher than AAV2 in animal and human nerve system and respiratory epithelial cell (AAV4 does not infect respiratory epithelial cell). The receptor of AAV1 is not yet clear. AAV6 is possibly a recombinant strain of AAV1 and AAV2, whose receptor is also unclear, but it is capable of binding with heparin, which provids affinity chromatograph conditions for its purification. It has been reported that the infection efficiency of AAV6 is 15-74 fold higher than AAV2 in respiratory epithelial cells of mice (J. V. 2001. 6615-6624. AAV6 vectors mediate efficient transfection of airway epithelial cells in mouse lungs-companed to that of AAV2 vectors).

“Capsid Change” Modification of Current AAV2 Vector (Construction of a Hybrid AAV Vector)

The “Capsid change” modification of current AAV2 vector is the simplest approach to obtain cell ecotropic AAV vectors of other five AAV serotypes. It is found in animal experiments that AAV1 exhibits generally higher transduction efficiency in tissues other than nerve tissue, such as muscle tissue and liver; while AAV5 has a better infection efficiency in retina, brain and islands of Langerhans compared with AAV2 vector (Terence Flotte, Anupam agarwal, Jianming Wang et al. 2001. Diabetes, 50: 515-520); wherein the infection efficiency of AAV1 in muscle tissue is 100-1000 times higher than AAV2 (Joseph E. Rabinowitz Fabienne Rolling Chengwen, 2002. J. Virol, 76: 791-801). The infection efficiencies of different AAV serotypes in liver and muscle tissue are in a descending sequence of 1, 5, 3, 2, 4; the infection efficiencies of different AAV serotypes in rat retina are in a descending sequence of 5, 4, 1, 2, 3. In a word, the study of any AAV serotype aside from AAV2 shall give AAV vectors having different infection specificity directing to different cell types, and as well expands the application field of AAV vectors.

Genomes in different AAV serotypes are highly homologous, but also have distinctive features, so that we could easily carry out “capsid change” modification of current AAV2 vector. That is, while keeping cis-element ITR (from AAV2) of current AAV vector unchanged, capsid proteins Caps are replaced by those from various AAV serotypes, resulted in obtaining cell ecotropic hybrid AAV vectors of serotypes of AAV1, AAV3, AAV4, AAV5, AAV6, respectively (i.e., ITR from AAV2, capsids respectively from AAV1, AAV3, AAV4, AAV5 and AAV6). AAV1, AAV3, AAV4, and AAV6 have a relatively high homology with AAV2. Thus, it is feasible to trans-packaging corresponding AAV hybrid vectors whose capsid is from AAV1, AAV3, AAV4, AAV5 or AAV6, and ITR is from the AAV2 by changing Cap while remaining Rep of AAV2.

Method for Production of AAV2 Vector

AAV2 genome consists of linear single-stranded DNA with a full length of approximately 4,800 bp, containing two inverted terminal repeats (ITR) of 145 bp length at the two ends, which are the origin of replication of AAV genome, and are associated with functions such as AAV replication, integration or packaging. The remaining portion of the genome could be divided into two functional regions: rep gene region and cap gene region. Rep gene has four forms of protein products: Rep78, Rep68, Rep52 and Rep40, which are essential regulatory proteins for AAV replication and virus gene expression, etc. Cap gene encodes three kinds of structural genes, VP1, VP2, VP3, which constitute the capsid of AAV vector together. The proteins encoded by rep and cap are all trans-acting proteins in AAV toxigenic replication. Thus, hybrid AAV vectors having the infection specificities of various serotypes are obtainable as long as cap proteins in various AAV serotypes are changed while ITR remains the same. Hence the vector cell strains having a large quantity of therapeutic genes and marker genes designed to package AAV2 vector is still useful, accordingly greatly simplifying the “capsid change” process of AAV vectors.

Traditional method for rAAV production is: co-transfecting rAAV vector plasmid and helper plasmid containing rep-cap gene into the cell, followed by helper virus infection, such as adenovirus or herpes simplex virus. After 2-3 days, the recombinant AAV (rAAV) and the used adenovirus or herpes simplex virus could be harvested from culture supernatant as well as pathologic cells. Adenovirus and herpes simplex virus could be deactiviated by heat treatment (under 55° for 30 minutes to 2 hours) without prejudice of AAV reactivity.

Although this method for production rAAV is relatively simple, disadvantages are also obvious. Firstly, double plasmids for co-transfection of cell and a large-scale preparation of plasmid DNA are required in each production of rAAV. The limitations of the transfection method, low transfection and co-transfection efficiencies render low titer of the resulted rAAV. Moreover, since it is still difficult to scale up to large-scale cell transduction with the transfection method, and thus, the method is unsuitable in mass production of rAAVs. Therefore, it becomes necessary to find a system and a method for mass production of rAAVs.

Yan Ziying, et al. filed an application entitled “Herpes simplex virus vector useful in packaging recombinant adeno-associated virus and use thereof” (Chinese Application No: 96 1 20549.0, Publication No: CN 1159480A), which introduces a method of constructing pHSV-AAV(+/−) by introducing rep-cap gene of AAV2 into a HSV1 amplicon vector plasmid. A mixed virus of wild type HSV-1 and pseudovirus containing rep-cap gene are obtainable by introducing the above plasmid into the cell in the presence of HSV-1 wild-type virus. The mixed virus has all helper functions of rAAV, including replication and packaging, but still fails to achieve a satisfactory result as expected due to low efficiency. Besides, Conway, et al. (Conway J E et al., J. Virol. 71: 8780-8789, 1997) have also reported a similar research. However, the ratio of the pseudovirus in the mixed virus is rather small (<10%), and thus, the helper functions thereof are limited. Furthermore, the ratio of pseudovirus to wild-type virus is not stable, which is unfavorable for quality control in mass production.

Wu Xiaobing, et al. have constructed full-function helper virus useful in simple and mass production of recombinant adeno-associated virus by introducing rep-cap gene into a HSV1 genome (Wu Xiaobing, et al., Process for preparing full-function helper virus used for production of recombinant adeno-associated virus and the usage thereof, Chinese Application No: 98120033.8). Abundant infectious rAAV virions are obtainable by infecting cells having been transfected with rAAV vector plasmid or cell strains stably carrying rAAV vector plasmid with HSV1-rc. The rAAV produced with this method is capable of introducing exogenous genes into mammalian cells and expressing the same (Wu Zhijian, Wu Xiaobing et al., Production of recombinant HSV having AAV vector packaging function, Chinese Science Bulletin, 1999,44 (5): 506-509; Wu Zhijian, Wu Xiaobing et al., a high-efficient system for production of recombinant adeno-associated virus vector, Science in China (Series C). 2001, 31(5): 423-430; WU Zhijian, WU Xiaobing, et al. Anovel and high efficient production system for recombinant adeno-associated virus vector, Science in China (Series C). 2002,45(1): 96-104; WU Zhijian, WU Xiaobing, et al. Gerreration of a recombinant herpes simplex virus which can provide packaging function for recombinant adeno-associated virus, Chinese Science Bulletin. 1999,44(8): 715-718).

Adeno-associated virus (AAV) vector develops the fastest and has become the most promising virus vector for gene therapy, owing to its safety, good stability, long term expression, wide infection spectrum, and capacity of infecting non-dividing cells, etc. AAV vector used to be constructed based on AAV serotype 2 (AAV2). However, the studies in the recent years show that the AAV2 infection efficiency is quite low in some tissues; in addition, the human body could produce neutralizating antibody against AAV infection, and about 85% of normal population develops the antibody against AAV2. This may affect the use of AAV2 vector in gene therapy.

There are six serotypes of AAV in nature, respectively named AAV1, AAV2, AAV3, AAV4, AAV5 and AAV6. The homology of the replificaton-associated proteins of Rep in the six AAV serotypes is comparatively high, but cap genes for expressing capsid proteins vary widely. The differences are responsible for many differences of the six AAV serotypes in areas such as infection specificity and antigenicity. Thus, AAV capsids of different serotypes could be used, so that AAV vectors have comparatively high infection efficiency in different human histiocytes. Moreover, when human body has produced a neutralization antibody directed to a specific AAV vector, another AAV vector could be used in order to increase infection efficiency of AAV vector.

### SUMMARY OF THE INVENTION

The recombinant adeno-associated virus vectors involved in the present invention refer to serotypes 1, 3, 4, 5, 6, i.e. rAAV-1, rAAV-3, rAAV-4, rAAV-5, rAAV-6. The present invention relates to methods for large-scale production, isolation and purification of the five serotypes of recombinant adeno-associated virus vectors and uses thereof.

The present invention does not relate to methods for large-scale production of recombinant adeno-associated virus vector of serotype 2 and uses thereof (i.e. rAAV-2). The relevant methods for large-scale production of recombinant adeno-associated virus vector of serotype 2 and uses thereof have been described in our prior application No: 99119039.4, entitled “Method for large-scale production of adeno-associated virus vector as well as use thereof”.

The present invention is based on Chinese Applications Nos. 99119039.4, 02117965.4, 99119038.6 and 99123723.4.

In our prior application “Method for large-scale production of adeno-associated virus vector and uses thereof” (Application No: 99119039.4; Publication No: CN 1252441A), a strategy of “one vector cell/one helper virus” for producing recombined adeno-associated virus vector of serotype 2 (rAAV-2) is provided.

The invention also uses the strategy of “one vector cell/one helper virus” for production, but the “one helper virus” will be HSV1-r2c1, HSV1-r2c3, HSV1-r2c4, HSV1-r2c5, HSV1-r2c6 respectively; corresponding to the used helper virus, the capsids of the resulted recombined adeno-associated virus vector will also have capsids of rAAV-1, rAAV-3, rAAV-4, rAAV-5, rAAV-6, respectively.

“One vector cell” in the present invention refers to a cell introduced with eukaryotic expression plasmid vector pSNAV as well as modified vector thereof. On said eukaryotic expression plasmid vector pSNAV, we have designed a gene expression cassette comprising ITR element of AAV and inserting sites for target gene. The detailed construction process and content of the eukaryotic expression plasmid vector pSNAV could be found in our prior-application (Application No: 99119038.6, Publication No: CN 1252450A), and later published referencee (Wu Zhijian, Wu Xiaobing, Hou Yunde, Construction of a series of adeno-associated virus vectors and study on β-galactosidase expression, Chinese Journal of Virology, 2000,16 (1), 1-6).

Various target genes could be inserted into multiple cloning sites, consequently producing rAAVs comprising different kinds of target genes. The type of the used helper viruses determines the serotypes of the rAAVs comprising different kinds of target genes.

The “cell introduced with eukaryotic expression vector pSNAV” in the present invention refers to various permissive cells of AAV and pSNAV. We have tried such subculturing cells as BHK Vero, CHO, 293, and various rodent and human histiocytes are proved to be infectionable by viruses such as AAV, HSV, etc. Permissive cells used herein refer to cells that could accept or endure infection and growth of some virus or organism.

To be adapted to the needs of our experiments, we have made a series of modification to the eukaryotic expression plasmid vector pSNAV involved in our prior application (Application No: 99119038.6). We have respectively replaced ITR elements of AAV-2 in pSNAV by ITR element of AAV-1, ITR element of AAV-3, ITR element of AAV-4, ITR element of AAV-5, ITR element of AAV-6, respectively constituting pSNAV-N1, pSNAV-N3, pSNAV-N4, pSNAV-N5, pSNAV-N6 (commonly known as: pSNAV-NX, wherein X can respectively refer to 1, 3, 4, 5, 6).

The present invention specifically relates to methods for large-scale production of five sterotypes of adeno-associated virus vectors as well as the use thereof, which all adopt the strategy of “one vector cell/one helper virus” for production.

Concerning “One Vector Cell”:

In rAAV-1 production, we infect vector cells with helper virus HSV1-r2c1 for mass production of recombinant viruses having rAAV-1 capsids, wherein one vector cell is introduced with eukaryotic expression plasmid vector pSNAV containing AAV2 ITR element, the other vector cell is introduced with eukaryotic expression plasmid vector pSNAV-N1 containing AAV1 ITR element. Experiments show that the use of the two kinds of cells do not have adverse effect on packaging and production of viruses, and the cells are both capable of packaging virions having AAV-1 capsids. The difference only lies in that ITR elements contained in gene expression cassettes of the two rAAV-1 virus particles are different: one containing AAV ITR element, named recombinant AAV2/1 hybrid virus; the other containing AAV1 ITR element, named recombinant AAV1 virus. Despite of high homology of the two ITR elements, they are still different.

In rAAV-3 production, we infect vector cells with helper virus HSV1-r2c3 for mass production of recombinant viruses having rAAV-3 capsids, wherein one vector cell is introduced with eukaryotic expression plasmid vector pSNAV containing AAV2 ITR element; the other vector cell is introduced with eukaryotic expression plasmid vector pSNAV-N3 containing AAV3 ITR element. Experiments show that the use of the two kinds of cells do not have adverse effect on packaging and production of viruses, and the cells are both capable of packaging virions having AAV-3 capsid. The difference only lies in that ITR elements contained in gene expression cassettes of the two rAAV-3 virus particles are different: one containing AAV2 ITR element, named recombinant AAV2/3 hybrid virus; the other containing AAV3 ITR element, named recombinant AAV3 virus. Despite of high homology of the two ITR elements, they are still different.

In rAAV-4 production, we infect vector cells with helper virus HSV1-r2c4 for mass production of recombinant viruses having rAAV-4 capsids, wherein one vector cell is introduced with eukaryotic expression plasmid vector pSNAV containing AAV2 ITR element; the other vector cell is introduced with eukaryotic expression plasmid vector pSNAV-N4 containing AAV4 ITR element. Experiments show that the use of the two kinds of cells do not have adverse effect on packaging and production of viruses, and the cells are both capable of packaging virions having AAV-4 capsids. The difference only lies in that ITR elements contained in gene expression cassettes of the two rAAV-4 virus particles are different: one containing AAV2 ITR element, named recombinant AAV2/4 hybrid virus; the other containing AAV4 ITR element, named recombinant AAV4 virus. Despite of high homology of the two ITR elements, they are still different.

In rAAV-5 production, we separately infect vector cells with helper virus HSV1-r2c5 for mass production of recombinant viruses having rAAV-5 capsids, wherein the vector cell is introduced with eukaryotic expression plasmid vector pSNAVcontaining AAV2 ITR element, and the resulted virus therefrom is named recombinant AAV2/5 hybrid virus. We do not use the vector cell introduced with eukaryotic expression plasmid vector pSNAV-5 containing AAV5 ITR element, since Rep of AAV2 is unable to identify ITR of AAV5 (John A. Chiorini, Sandra Afione and Robert M. Kotin, Adeno-associated virus type 5 Rep protein cleaves a unique terminal resolution site compared with other AAV serotypes, Journal of Virology, 1999, 4293-4298)(Markus Hildinger, James m. Wilson et al. Hybrid vectors based on adeno-associated virus serotypes 2 and 5 for muscle-directed gene transfer, Journal of Virology, 2001, 6199-6203).

In rAAV-6 production, we infect vector cells with helper virus HSV1-r2c6 for mass production of recombinant viruses having rAAV-6 capsids, wherein one vector cell is introduced with eukaryotic expression plasmid vector pSNAV containing AAV6 ITR element; the other vector cell is introduced with eukaryotic expression plasmid vector pSNAV-N6 containing AAV6 ITR element. Experiments show that the use of the two kinds of cells do not have adverse effect on packaging and production of viruses, and the cells are both capable of packaging virions having AAV-6 capsid. The difference only lies in that ITR elements contained in gene expression cassettes of the two rAAV-4 virus particles are different: one containing AAV2 ITR element, named recombinant AAV2/6 hybrid virus; the other containing AAV6 ITR element, named recombinant AAV6 virus. Despite of high homology of the two ITR elements, they are still different.

“One Helper Virus”

“one helper virus” refers to a helper virus used in infecting “one vector cell”, and is capable of inducing the vector cell to produce rAAV. The present invention uses recombinant herpes simplex virus type 1 (rHSV-1).

The present invention describes five recombinant herpes simplex viruses (respectively HSV1-r2c1, HSV1-r2c3, HSV1-r2c4, HSV1-r2c5, HSV1-r2c6, generalized as HSV1-rXcY). Their commonness lies in that their genomes are all inserted with (AAV2) rep gene, and the difference lies in that their genomes are respectively inserted with cap genes of serotype 1 (AAV1), 3(AAV3), 4(AAV4), 5(AAV5), 6(AAV6). That is, a combination of rep gene of AAV2 with cap gene of AAV1 is inserted into a recombinant herpes simplex virus genome to give recombinant herpes simplex virus HSV1-r2c1, a combination of rep gene of AAV2 with cap gene of AAV3 is inserted into a recombinant herpes simplex virus genome to give recombinant herpes simplex virus HSV1-r2c3, a combination of rep gene of AAV2 with cap gene of AAV4 is inserted into a recombinant herpes simplex virus genome to give recombinant herpes simplex virus HSV1-r2c4, a combination of rep gene of AAV2 with cap gene of AAV5 is inserted into a recombinant herpes simplex virus genome to give recombinant herpes simplex virus HSV1-r2c5, a combination of rep gene of AAV2 with cap gene of AAV6 is inserted into a recombinant herpes simplex virus genome to give recombinant herpes simplex virus HSV1-r2c6.

The recombinant herpes simplex viruses are respectively used to infect vector cells, and the infected vector cells are capable of expressing Cap proteins of AAV1, 3, 4, 5, 6, respectively as well as expressing Rep protein of AAV2. The recombinant herpes simplex viruses (rHSV1-r2c1, rHSV1-r2c3, rHSV1-r2c4, rHSV1-r2c5, rHSV1-r2c6) are used as helper viruses in infecting cells having a gene expression cassette comprising ITR of AAV2 and gene sequences of exogenous genes (ITR—exogenous genes—ITR), respectivtly producing recombinant AAV vectors (rAAVs) having capsids of AAV serotypes 1, 3, 4, 5, 6. The type of AAV capsid protein Cap determines tissue infection specificity and infection efficiency of AAV. Using certain changed serotype capsid protein may produce a rAAV vector having highly sufficient in infecting some specific tissues, and accordingly increase effectiveness and security of rAAV vector in gene therapy.

The present invention utilize the same principle for constructing HSV1-rc as disclosed by Wu Xiaobing, et al. (Wu Xiaobing et al., Process for preparing full-function helper virus used for production of recombinant adeno-associated virus and uses thereof, Chinese Application No: 98120033.8. Wu Zhijian, Wu xiaobing et al., Chinese Science Bulletin. 1999,44 (5): 506-509. Wu Zhijian, Wu Xiaobing et al., Science in China (Series C). 2001, 31(5): 423-430. WU Zhijian, WU Xiaobing, et al. Science in China (Series C). 2002, 45(1): 96-104. WU Zhijian, WU Xiaobing, et al. Chinese Science Bulletin. 1999,44(8): 715-718). Based on the existing AAV2 full-function helper virus and by means of gene recombination of HSV1-rc, we may obtain helper viruses HSV1-r2c1, HSV1-r2c3, HSV1-r2c4, HSV1-r2c5, HSV1-r2c6 having the function of producing virus vector of AAV serotypes 1, 3, 4, 5, 6. The invention is characterized in that a copy of rep-cap gene (about 4 kb with no limited direction) respectively from AAV2 (rep) and AAV1, 3, 4, 5, 6 (cap) is inserted into HSV1 genome. In the five recombinant herpes simplex viruses constructed in the present invention, rep-cap gene is respectively inserted into XbaI sites of HSV1 UL2 gene (encoding uracil DNA glycosylases) or HSV1 UL44 gene (encoding glycoprotein C), or inserted into other nonessential gene regions of HSV1 gene by a homologous recombination method. Nonessential genes refer to genes that are not essential for proliferation and passage of HSV1 in cells cultured in vitro. In other words, the insertion of exogenous genes into the nonessential genes would not affect normal replication and propagation of HSV1. These recombinant HSV1 viruses could all propagate and stably passaged in HSV1 sensitive cells (such as BHK-21, Vero, Cho, 293). The five herpes simplex viruses constructed in the present invention could be used in infecting cells transfected by rAAV vector plasmid or cell strains steadily carrying rAAV vector plasmid, consequently producing infectious rAAV virions having capsids of AAV1, 3, 4, 5, 6, respectively.

The production of the several helper viruses used in the present invention is based on modification of a set of cosmid Set C comprising whole genome of HSV1 (including a total of five cosmids such as cos6, cos14, cos28, cos48, cos56, Cunningham, C. and A. J. Davison 1993 A cosmid-base system for constructing mutants of Herpes Simplex Virus Type 1. Virology 197: 116-124).

In the present invention, cap genes of AAV1, 3, 4, 5, 6 are respectively linked with rep gene of AAV2 to form DNA segments of rep2cap1, rep2cap3, rep2cap4, rep2cap5, rep2cap6 (see FIG. 1, 2, 3, 4, 5), then the five DNA segments are respectively loaded into HSV1 genome, giving recombinant HSV1 that express cap proteins of AAV1, 3, 4, 5, 6, and rep protein of AAV2: HSV-r2c1, HSV-r2c3, HSV-r2c4, HSV-r2c5, HSV-r2c6, which are subsequently used to respectively infect cells being transfected with rAAV vector plasmid or cell strains steadily carrying rAAV vector plasmid, and thereby to produce infectious rAAV virions respectively containing nucleocapsids of AAV1, 3, 4, 5, 6. The method for production of DNA segments of rep2cap1, rep2cap3, rep2cap4, rep2cap5, rep2cap6 DNA segments may be one out of the following methods: (1) rep gene of AAV2 is respectively linked with cap genes of AAV1 AAV2, AAV3, AAV4 and AAV5, AAV6; (2) under the premise of not interfering the packaging function of AAV2 Rep protein for AAV2 ITR, portions from rep genes (in downstream of the whole rep gene, i.e. 3′ end) of AAV1 AAV2, AAV3, AAV4 and AAV5 are used to replace corresponding portions of rep gene of AAV2, and in the meantime, cap genes of AAV1, AAV3, AAV4, AAV5 and AAV6 are used to replace cap gene of AAV2. Such partial replacement of rep gene could sometimes enhance packaging efficiency and output of rAAVs.

In addition, the inserted segments (rep and cap) having the function of producing AAV vectors of serotype 1, serotype 3, serotype 4, serotype 5, serotype 6 in helper viruses may also be from the same serotype, i.e. rep and cap genes are both from AAV1, AAV3, AAV4, AAV5 or AAV6.

Rep and cap segments may be inserted into the same position in HSV1 genome in single copy form, or be inserted into different positions in HSV1.

Rep and cap segments may also be inserted into the same position in HSV1 genome in two or more-copy form, or be inserted into different positions in HSV1.

The present invention is also applicable to the production of helper viruses of AAV vectors of other serotyeps aside from AAV1, AAV2, AAV3, AAV4, AAV5, and AAV6, such as AAV7, AAV8.

The five DNA segments are respectively loaded into HSV1 genome to give five strains of recombinant HSV1 capable of respectively expressing cap protein of AAV1, 3, 4, 5, 6 and rep protein of AAV2 at the same time: HSV1-r2c1 is capable of simultaneously expressing cap protein of AAV1 and rep protein of AAV2; HSV1-r2c3 is capable of simultaneously expressing cap protein of AAV3 and rep protein of AAV2; HSV1-r2c4 is capable of simultaneously expressing cap protein of AAV4 and rep protein of AAV2; HSV1-r2c5 is capable of simultaneously expressing cap protein of AAV5 and rep protein of AAV2; HSV1-r2c6 is capable of simultaneously expressing cap protein of AAV6 and rep protein of AAV2. When the five herpes simplex viruses are used as helper viruses to respectively infect cells being transfected with rAAV vector plasmids or cell strains steadily carrying rAAV vector plasmid, they shall accordingly produce infectious rAAV virions respectively containing nucleocapsids of serotypes 1, 3, 4, 5, 6.

The features of the five strains of recombinant herpes simplex viruses (generalized as HSV1-rXcY) are detailed as follows:

(1) HSV1-r2c1:

A recombinant herpes simplex virus (HSV1), characterized in that a DNA sequence is inserted into the genome, which sequence having a nucleotide sequence as shown in SEQ ID NO.1 (means rep2cap1) or a homologous sequence thereof. Wherein the DNA sequence SEQ ID NO.1 is inserted into XbaI site of UL2 gene of HSV1 genome. Rep2cap1 nucleotide sequence segment is inserted into XbaI site of UL2 gene of COS6 of Sect C.

Besides, the DNA sequence SEQ ID NO.1 could also be inserted into other nonessential gene zones by inserting XbaI site of UL44 gene in HSV1 or by a homologous recombination method, such as replacing nonessential gene tk in HSV1 by SEQ ID NO.1 with a homologous arm method. Rep2cap1 nucleotide sequence segment is inserted into XbaI site of UL44 gene of COS56 of Sect C.

(2) HSV1-r2c3:

A recombinant herpes simplex virus (HSV1), characterized in that a DNA sequence is inserted into the genome, which sequence having a nucleotide sequence as shown in SEQ ID NO.2 (means rep2cap3) or a homologous sequence thereof. Wherein the DNA sequence SEQ ID NO.2 is inserted into XbaI site of UL2 gene of HSV1 genome. Rep2cap3 nucleotide sequence segment is inserted into XbaI site of UL2 gene of COS6 of Sect C.

Besides, the DNA sequence SEQ ID NO.2 could also be inserted into other nonessential gene zones by inserting XbaI site of UL44 gene in HSV1 or by a homologous recombination method, such as replacing nonessential gene tk in HSV1 by SEQ ID NO.2 with a homologous arm method. Rep2cap3 nucleotide sequence segment is inserted into XbaI site of UL44 gene of COS56 of Sect C.

(3) HSV1-r2c4:

A recombinant herpes simplex virus (HSV1), characterized in that a DNA sequence is inserted into the genome, which sequence having a nucleotide sequence as shown in SEQ ID NO.3 (means rep2cap4) or a homologous sequence thereof. Wherein the DNA sequence SEQ ID NO.3 is inserted into XbaI site of UL2 gene of HSV1 genome. Rep2cap4 nucleotide sequence segment is inserted into XbaI site of UL2 gene of COS6 of Sect C.

Besides, the DNA sequence SEQ ID NO.3 could also be inserted into other nonessential gene zones by inserting XbaI site of UL44 gene in HSV1 or by a homologous recombination method, such as replacing nonessential gene tk in HSV1 by SEQ ID NO.3 with a homologous arm method. Rep2cap4 nucleotide sequence segment is inserted into XbaI site of UL44 gene of COS56 of Sect C.

(4) HSV1-r2c5:

A recombinant herpes simplex virus (HSV1), characterized in that a DNA sequence is inserted into the genome, which sequence having a nucleotide sequence as shown in SEQ ID NO.4 (means rep2cap5) or a homologous sequence thereof. Wherein the DNA sequence SEQ ID NO.4 is inserted into XbaI site of UL2 gene of HSV1 genome. Rep2cap5 nucleotide sequence segment is inserted into XbaI site of UL2 gene of COS6 of Sect C.

Besides, the DNA sequence SEQ ID NO.4 could also be inserted into other nonessential gene zones by inserting XbaI site of UL44 gene in HSV1 or by a homologous recombination method, such as replacing nonessential gene tk in HSV1 by SEQ ID NO.4 with a homologous arm method. Rep2cap5 nucleotide sequence segment is inserted into XbaI site of UL44 gene of COS56 of Sect C.

(5) HSV1-r2c6:

A recombinant herpes simplex virus (HSV1), characterized in that a DNA sequence is inserted into the genome, the sequence having a nucleotide sequence as shown in SEQ ID NO.5 (means rep2cap6) or a homologous sequence thereof. Wherein the DNA sequence SEQ ID NO.5 is inserted into XbaI site of UL2 gene of HSV1 genome. Rep2cap6 nucleotide sequence segment is inserted into XbaI site of UL2 gene of COS6 of Sect C.

Besides, the DNA sequence SEQ ID NO.5 could also be inserted into other nonessential gene zones by inserting XbaI site of UL44 gene in HSV1 or by a homologous recombination method, such as replacing nonessential gene tk in HSV1 by SEQ ID NO.5 with a homologous arm method. Rep2cap6 nucleotide sequence segment is inserted into XbaI site of UL44 gene of COS56 of Sect C.

The present invention discloses methods for the preparation of five strains of recombinant herpes simplex viruses, comprising constructing DNA segments of SEQ ID NO: 1, SEQ ID NO: 2, SEQ ID NO: 3, SEQ ID NO: 4 or SEQ ID NO: 5, and inserting the DNA segments into a herpes simplex virus genome using a gene engineering method to give a recombinant herpes simplex virus; or inserting another DNA sequence homologous to a DNA segment of SEQ ID NO: 1, SEQ ID NO: 2, SEQ ID NO: 3, SEQ ID NO: 4 or SEQ ID NO: 5 to give a recombinant herpes simplex virus HSV-rXcY having the same or similar functions. Meanwhile, the uses of the five strains of recombinant herpes simplex viruses are also put forward in the present invention.

Source biomaterials for constructing five strains of recombinant herpes simplex viruses in the present invention include Set C cosmid, etc.

Set C cosmid is composed of five cosmids sequentially loaded with HSV1 whole genome: cos6, cos14 cos28, cos48, cos56, a gift from Davision A J (Conningham C, Davision A J. Virology, 1993, 197: 116-124). (For sequences of cos6, cos14 cos28, cos48, cos56, please see Seq6, Seq7, Seq8, Seq9, Seq10). The ends of each HSV1 genome segment load in the cosmid set shall overlap with the ends of HSV1 segments in another cosmid. This serves as the foundation for homologous recombination of the five HSV1 genome segments in cells and corresponding production of recombinant HSV1 therefrom (see FIG. 8). Nonessential gene UL2 on cos6 in Sect C and nonessential gene UL44 of HSV1 on cos56 have a single XbaI cuting point for inserting exogenous genes thereto, and producing recombinant HSV virus inserting with exogenous genes by means of recombination of five cosmids.

Background of the three virus strains AAV1, AAV3, AAV4: virus strains with ATCC accession numbers respectively being ATCC VR645, ATCC VR681, ATCC VR646.

AAV5 virus strain: its source referring to reference (Bantel-Schaal U, Zur Hausen H. Virology 1984, 134: 52-63).

AAV6 virus strain: its source referring to reference (Rutledge, E. A., Halbert, C. L. and Russell, D. W. J. Virol. 1998, 72: 309-319).

SSV9: plasmid containing rep and cap genes of AAV2 (Du B, Wu P, Boldt-Houle D M, Terwilliger E F Gene Ther 1996, 3: 254-61).

Relevant Patents

Wu Xiaobing, et al. Construction of recombinant herpes simplex virus based on cosmid and use thereof, Chinese Application No: 98101753.3, Publication No: CN 1234441A;

Wu Xiaobing, et al.: Method for preparing full-function helper virus used in production of recombinant adeno-associated virus and use thereof, Chinese Application No: 98120033.8, Publication No: CN 1243878A.

Wu Xiaobing, et al.: Method for large-scale production of recombinant adeno-associated virus and use thereof, Chinese Application No: 99119039.4, Publication No: CN 1252441A.

Wu Xiaobing, et al.: Method for fast and efficient isolation and purification of recombinant adeno-associated virus and use thereof, Chinese Application No: 99123723.4, Publication No: CN 1272538A.

Method for Preparing Five Strains of Recombinant Herpes Simplex Viruses:

The strains are prepared by substantially the same strategy and method as used in the preparation of HSV1-lacZ100 recombinant virus. The method comprises inserting lacZ gene into XbaI site of cos6 and recombining five cosmids to give HSV1-lacZ100 recombinant virus (Wu Xiaobing, et al.: Construction of recombinant herpes simplex virus based on cosmid and use thereof, Chinese Application No: 98101753.3).

After rep (AAV2) and cap (AAV1, AAV3, AAV4, AAV5, AAV6) genes are respectively obtained from respective virus genome templates by PCR using upstream and downstream primers, they are subjected to restriction enzyme digestion and ligation to respectively give corresponding r2c1, r2c3, r2c4, r2c5 and r2c6 gene segments, which have XbaI sites on both ends (see FIG. 1, 2, 3, 4, 5).

In r2c5 gene segment, rep2 is a hybrid rep gene with one portion of rep gene of AAV5 and one portion of rep gene of AAV2, i.e. the rep2 is a hybrid gene of partial rep5 and partial rep2. The preparation process is as follows: after one portion of rep2 is obtained from AAV2 virus genome template by a couple of rep primers for AAV2 with PCR method, and another portion of rep5 is obtained from AAV5 virus genome template by a couple of rep primers for AAV5 with PCR method, these portions are subjected to restriction enzyme digestion and ligation to give a hybrid gene containing partial rep5 and partial rep2.

HSV 1 genome segments loaded in cos6 and cos56 cosmids each have a XbaI single enzyme cutting site, respectively locating whithin nonessential genes UL2 and UL44, generally useful for inserting exogenous genes. After XbaI enzyme cutting, gene segments r2c1, r2c3, r2c4, r2c5 and r2c6 are inserted into XbaI site of cos6 to construct recombinant cosmids cos6-r2c1ΔUL2, cos6-r2c3ΔUL2, cos6-r2c4ΔUL2, cos6-r2c5ΔUL2, cos6-r2c6ΔUL2, respectively (generalized as cos6-rXcYΔUL2, see FIG. 6). Any one of cos6-r2c1ΔUL2, cos6-r2c3ΔUL2, cos6-r2c4ΔUL2, cos6-r2c5ΔUL2, cos6-r2c6ΔUL2 is then respectively equimolarly mixed with cos14, cos28, cos48, cos56, and, removing cos framework with PacI enzyme cutting and cotransfecting BHK-21 cells with liposome, so that five HSV1 segments undergo homologous recombinations in cells and respectively give such recombinant viruses as HSV1-r2c1, HSV1-r2c3, HSV1-r2c4, HSV1-r2c5, HSV1-r2c6. Five days later, cells begin exhibit pathology, collecting the culture supernatant after complete pathology of the cells, then centrifugating at 2000 r/min for 5 min, and dividing the supernatant in aliquots for storage under −20°. The probability of producing recombinant HSV1 virus containing target DNA segment is as high as 50˜100%. It is easy to pure recombinat virus through further plaque screening.

Similarly, it is also feasible to obtain recombinant viruses having the same functions as the recombinant viruses HSV1-r2c1, HSV1-r2c3, HSV1-r2c4, HSV1-r2c5, HSV1-r2c6 by respectively inserting such gene segments as r2c1, r2c3, r2c4 and r2c5 into XbaI site of cos56. The preparation process is as follows: after XbaI enzyme cutting, gene segments r2c1, r2c3, r2c4, r2c5 and r2c6 are respectively inserted into XbaI site of cos56, in order to respectively construct recombinant cosmids cos56-r2c1ΔUL44, cos56-r2c3ΔUL44, cos56-r2c4ΔUL44, cos56-r2c5ΔUL44, cos56-r2c6ΔUL44 (generalized as cos56-rXcYΔUL44, see FIG. 7). Any one of cos56-r2c1ΔUL44, cos56-r2c3ΔUL44, cos56-r2c4ΔUL44, cos56-r2c5ΔUL44, cos56-r2c6ΔUL44 is then respectively equimolarly mixed with cos6, cos14, cos28, cos48, and, removing cos framework with PacI enzyme cutting and cotransfecting BHK-21 cells with liposome, so that five HSV1 segments undergo homologous recombinations in cells and respectively give such recombinant viruses as HSV1-r2c1, HSV1-r2c3, HSV1-r2c4, HSV1-r2c5, HSV1-r2c6: five days later, cells begin exhibit pathology, collecting the culture supernatant after complete pathology of the cells, then centrifugating at 2000 r/min for 5 min, and dividing the supernatant in aliquots for storage under −20°. The probability of producing recombinant HSV1 virus containing target DNA segment can also be as high as 50˜100%. It is easy to pure recombinat virus through further plaque screening.

In addition, it is also feasible to obtain recombinant viruses having the same functions as the recombinant viruses HSV1-r2c1, HSV1-r2c3, HSV1-r2c4, HSV1-r2c5, HSV1-r2c6, etc. by inserting such gene segments as r2c1, r2c3, r2c4, r2c5, r2c6 etc. into HSV1 genome by ways of recombination of homologous arms, transposons, site-directed insertion, random insertion, etc.

Likewise, recombinant viruses such as HSV1-r2c1, HSV1-r2c3, HSV1-r2c4, HSV1-r2c5, HSV1-r2c6, etc. described in the present invention may also be inserted with other DNA sequences which are homologous to DNA segments of SEQ ID NO.1, SEQ ID NO.2, SEQ ID NO.3, SEQ ID NO.4 or SEQ ID NO.5. “Other homologous DNA sequences” refer to other non-SEQ ID NO. 1, -SEQ ID NO.2, -SEQ ID NO.3, -SEQ ID NO.4 or -SEQ ID NO.5 sequences, but have certain DNA sequence homology therewith, and could also act the functions of helper viruses of AAV vectors.

Five Strains of Recombinant Herpes Simplex Viruses are Used to Respectively Prepare rAAV1, 3, 4, 5, 6 of AAV Serotypes 1, 3, 4, 5, 6:

Five strains of recombinant herpes simplex viruses are used to respectively prepare five types of recombinant AAV containing capsid proteins of AAV serotypes 1, 3, 4, 5, 6: rAAV1, rAAV3, rAAV4, rAAV5, rAAV6.

Among AAV1, 3, 4, 5, 6, rep of AAV5 and rep of AAV2 have the greatest difference. Thus it is desirable to replace a portion of rep of AAV2 with a portion of rep of AAV5, so as to effectively guarantee gene replication of AAV5 and packaging of rAAV5 virus (Yoon, M, D. Smith, P. Ward, F, et al. 2001. J. Virol. 75: 3230-3239). HSV1-r2c5 in the present invention retains 5′ end of rep gene of AAV2 in a length of about 860 bp (in BamHI site), to be used in infecting a AAV packaging cell strain containing ITR (AAV2)-exogenous gene-ITR (AAV2), and thereby to give rAAV5 virus containing AAV serotype 5. As indicated earlier, aside from the method of replacing a portion of rep of AAV2 with a portion of rep of AAV5, the complete use of rep of AAV2 could also package a AAV5 hybrid virus having a relatively high titer (Dirk Grimmk, Mark A. Kay, et al. Pre-clinical in vivo evaluation of pseudotyped adeno-associated virue vectors for liver gene therapy, Blood, prepublished online Jun. 5, 2003, DOI 10.1182/blood-2003-02-0495).

Except for AAV5, AAV1, 3, 4, 6 have a comparatively great homology with AAV2 in rep. Thus, rep genes of AAV in the construction of HSV1-r2c1, HSV1-r2c3, HSV1-r2c4, HSV1-r2c6 in the present invention are all substantially from AAV2, in which the rep gene is used to respectively infect AAV packaging cell strains containing ITR (AAV2)-exogenous gene-ITR (AAV2), and thereby to give rAAV1, 3, 4, 6 containing AAV serotypes 1, 3, 4, 6.

The process for constructing AAV packaging cell strain is as follows: constructing plasmid vector pSNAV containing “ITR (AAV2)-exogenous gene-ITR (AAV2)” and resistance genes such as neo^{r} (Application No: 99119038.6, Publication No: CN 1252450A), transfecting the vector into a HSV1-sensitive passaging cell strain, such as BHK-21, screening the resistance cell strain with G418 to give AAV packaging cell strain. Then, it is infected by a rHSV1 helper virus containing rep-cap of AAV to finally give rAAV vector (the process referring to: Construction of recombinant herpes simplex virus based on cosmid and use thereof, Chinese Application No: 98101753.3; Wu Xiaobing, et al.: Method for preparing full-function helper virus used in production of recombinant adeno-associated virus and use thereof, Chinese Application No: 98120033.8).

We have made a series of modification to plasmid vector pSNAV. Specifically, we have respectively replaced ITR element of AAV-2 in pSNAV by ITR element of AAV-1, ITR element of AAV-3, ITR element of AAV-4, ITR element of AAV-5, ITR element of AAV-6, to respectively construct pSNAV-N1, pSNAV-N3, pSNAV-N4, pSNAV-N5, pSNAV-N6 (generalized as: pSNAV-NX, wherein X can respectively refer to 1, 3, 4, 5, 6). The various plasmid vectors are then transfected into HSV1-sensitive passaging cell strain, such as BHK-21, and the resistentce cell strain is then screened with G418 to give AAV packaging cell strain. Then, it is subsequently infected by rHSV1 helper virus containing rep-cap of AAV to finally give rAAV vector.

Large-scale Production of Five Serotypes of Recombinant Adeno-associated Virus Vectors:

Production of rAAV-1: Infecting vector cells having been introduced with pSNAV or pSNAV-N1 with helper virus HSV1-r2c1 for mass production of rAAV-1 viruses.

Production of rAAV-3: Infecting vector cells having been introduced with pSNAV or pSNAV-N3 with helper virus HSV1-r2c3 for mass production of rAAV-3 viruses.

Production of rAAV-4: Infecting vector cells having been introduced with pSNAV or pSNAV-N4 with helper virus HSV1-r2c4 for mass production of rAAV-4 viruses.

Production of rAAV-5: Infecting vector cells having been introduced with pSNAV or pSNAV-N5 with helper virus HSV1-r2c5 for mass production of rAAV-5 viruses.

Production of rAAV-6: Infecting vector cells having been introduced with pSNAV or pSNAV-N6 with helper virus HSV1-r2c6 for mass production of rAAV-6 viruses.

Our prior-filed application “Method for fast and efficient isolation and purification of recombinant adeno-associated virus and use thereof” (Patent Application No: 99123723.4, Publication No: CN 1272538A) has disclosed a novel method for isolation, purification and condensation of recombinant adeno-associated virus. Similarly, the isolation and purification of the five serotypes of recombinant adeno-associated virus vectors of the application are also based on the method of isolation and purification as disclosed in the prior-invention. To be specific:

1. The steps are as follows:
1) adding chloroform to break cells, deactivate HSV helper viruses and to denature and precipitate a great many cell proteins;
2) treating the cell lysis solution with DNaseI and RNase to degrade the nucleic acid;
3) adding NaCl to accelerate isolation of rAAV from cell debris, and removing the cell debris with centrifugation;
4) precipitating rAAV with PEG/NaCl;
5) using chloroform to extract and remove other proteins and the residual PEG
6) removing salts via dialysis; and
7) further purifying rAAV via density gradient centrifugation or affinity chromatography.

2. The specific operations are indicated as follows:
1) Large-scale production of rAAV: various HSV1-rXcY mentioned in the present invention are used as helper viruses for infecting corresponding vector cells, and as long as cells exhibit complete CPE change and float up (about 48˜72 hr), cell cultures (cells as well as culture media) are harvested as crude lysis solution, measuring the volume thereof.
2) Deactivation of helper viruses and cell lysis: the treatment of raw material solution (i.e. crude lysis solution) with chloroform could achieve dual purposes of deactivating helper virus HSV1-rXcY and lysing cells, but has no influence on AAVs. Infectious herpes simplex virus particles have a lipid bilayer outer membrane as well as multiple virus glycoproteins veneered therein, wherein the membrane is essential in infecting cells by HSV viruses. Chloroform is capable of dissolving lipid, and denaturing abundant proteins. The treatment with chloroform could 100% deactivate HSV viruses, and as well lyse cell membrane and nuclear membrane with high efficiency. As AAV particles have chloroform-resistant activity, chloroform treatment has no influence on structure and infection activity of the particles.
3) Removal of cell debris and denatured proteins: solid sodium chloride is added into cell lysis solution to a final concentration of 1.0˜1.2 mol/L, accompanied by stirring for dissolution. The mixture is then centrifugated at 11000 g for 10˜15 min. Afterwards, the supernatant is transferred into a clean medium-sized conical flask for volume evaluation. The centrifugated precipitation and lower layer chloroform are eventually removed. The addition of sodium chloride could accelerate isolation of AAV particles from cell debris, and is also essential for precipitation of AAVs with polyglycol in the succeeding step.
4) Solid polyglycol 8000 is added to the supernatant to a final concentration of 6˜12%, accompanied by stirring for dissolution. The mixture is disposed at 4° for 1 hour to overnight. The mixture is then centrifugated at 12000 g for 10˜15 min. Afterwards, the supernatant is decanted into another clean conical flask, letting the supernatant draining away as much as possible. The precipitation is then dissolved with an appropriate amount of PBS²⁺, and Dnasel and RNase are added to digest residual DNA and RNA aside from AAV particles. Equiareal chloroform is added for extraction, and the mixture is centrifugated at 12000 g for 5 min. The upper aqueous phase is carefully taken out under sterile operation, and transferred into a sterile pipe. The solution is the very concentrated and purified rAAV solution.
5) rAAV obtained by the above-mentioned method could reach a purity degree of >99%. As for the rAAVs, prepared from 2×10⁹ cells (five 110×288 mm roller bottles) crude lysis solution, titer could attain 10^{14˜15} particles/ml, infection titer could attain >10^{12˜13} TU/ml. The recovery rate of rAAV is >90%. The obtained rAAV is useful in in vitro experiments and animal experiments, and upon further purification, clinical rAAV product could be obtained therefrom.
6) The double aqueous-phase extraction could be used for further purification of the virus solution. The solution is firstly subjected to PEG/salt system or PEG/Dex system, eventually PEG and salt are removed with dialysis, and the solution is sterilized by ultrafiltration.
7) For further fine purification, the methods could also be applied: column chromatography (including molecular sieve chromatography, affinity chromatography) or cesium chloride ultracentrifugation as well as dialysis, ultrafiltration, etc.

After the crude purification, the purity of purified AAV vector virus is over 60%, the content of contaminated proteins is less than 40%. Thus treated AAV vector viruses are free of most of other proteins and lipid from cells, and make them easy for further fine purification, and accordingly prepare AAV vectors consistent with clinical experimental standards.

The further fine purification of the obtained rAAV liquid comprises: passing the rAAV solution through an ion exchange column which has been balanced by a buffer, balancing the ion exchange column using a buffer again, then eluting the ion exchange column with a salt-containing buffer and collecting the elution peaks; passing the collected elution peaks through a molecular sieve column which has been balanced by a buffer, then washing the column with a buffer again to give a further purified rAAV; the specific operations are as follows:

The ion exchange column in the present invention can be selected from QFF column (Q Sepharose Fast Flow column, manufactured by Amersham Pharmacia), etc. The molecular sieve may be selected from S200 column (Sephacryl S-200 High Resolution column, manufactured by Amersham Pharmacia), etc.

The above obtained rAAV solution are loaded to a QFF column having been balanced by buffer, which is then further balanced by buffer, and subsequently, using eluting buffer with salt (may be selected from NaCl, etc, possibly a final concentration of 1M NaCl) and to collect the elution peaks.

The collected elution peaks are loaded to a S200 column having been balanced by buffer, and is further washed with buffer for elution, eventually the elution peaks are collected and are further purified to give clinical grade of rAAV.

Our study has found that the use of five strains of herpes simplex viruses to respectively infect cells (such as BHK cell) untransfected by AAV vector DNAs may also produce abundant AAV particles. The difference only lies in that the virus particles are empty capsids. It shows that in the formation of virus particles from AAVs, empty capsids are pre-packaged prior to packaging genome DNA into capsomeres.

### BRIEF DESCRIPTION OF THE DRAWINGS

FIG. 1 is a map of rep2cap1, wherein the main body of rep gene is from AAV2 (a length of about 1721 bp), and a small rep segment at 3′ end is from AAV1 (a length of about 280 bp); cap gene is entirely from AAV1 (a length of about 2210 bp).

FIG. 2 is a map of rep2cap3, wherein rep gene is entirely from AAV2; the main body of cap gene is from AAV3 (a length of about 2040 bp), and a small segment at 5′ end is from AAV2 (a length of about 30 bp).

FIG. 3 is a map of rep2cap4, wherein the main body of rep gene is from AAV2 (a length of about 1721 bp), and a small rep segment at 3′ end is from AAV4 (a length of about 280 bp); the main body of cap gene is from AAV4 (a length of about 2170 bp), and a small segment at 3′ end is from AAV2 (a length of about 160 bp).

FIG. 4 is a map of rep2cap5, wherein 5′ end of rep gene is from AAV2 (a length of about 860 bp), and 3′ end is from AAV5 (a length of about 1122 bp); cap gene is entirely from AAV5 (a length of about 2170 bp).

FIG. 5 is a map of rep2cap6, wherein the main body of rep gene is from AAV2 (a length of about 1721 bp), and a small segment at 3′ end is from AAV6 (a length of about 280 bp); cap gene is entirely from AAV6 (a length of about 2210 bp).

FIG. 6 is a map of cos6-rXcYΔUL2, wherein rXcY could respectively be r2c1, r2c3, r2c4, r2c5, r2c6 without any limitation on the directions.

FIG. 7 is a map of cos56-rXcYΔUL44, wherein rXcY could respectively be r2c1, r2c3, r2c4, r2c5, r2c6 without any limitation on the directions.

FIG. 8 is a map of Set C composed of cos6, cos28, cos14, cos56, cos48. After cut cos framework from Set C by Pac I, it infects cells and goes through homologous recombination to give HSV1 virus, wherein UL2 gene of HSV1 in cos6 and UL44 gene of HSV1 in cos56 separately have a XbaI site for inserting exogenous genes.

FIG. 9 is a map of pSNAV-GFP. GFP (green fluorescent protein) gene is initiated by an immediate early promoter of human CMV virus, and poly A is from SV40 virus. The two ends of GFP expression cassette are ITRs (inverted terminal repeats) of AAV2. The recombinant HSV viruses in the present invention: HSV-r2c1, HSV-r2c3, HSV-r2c4, HSV-r2c5, HSV-r2c6 are used to respectively infect cell strains having been transfected with pSNAV-GFP, and accordingly produce AAV vectors carrying reporter gene GFP expressed by AAV serotypes of 1, 3, 4, 5, 6.

FIG. 10 is a map of pSNAV-N1, wherein ITR is an ITR element of AAV-1.

FIG. 11 is a map of pSNAV-N3, wherein ITR is an ITR element of AAV-3.

FIG. 12 is a map of pSNAV-N4, wherein ITR is an ITR element of AAV-4.

FIG. 13 is a map of pSNAV-N5, wherein ITR is an ITR element of AAV-5.

FIG. 14 is a map of pSNAV-N6, wherein ITR is an ITR element of AAV-6.

FIG. 15 shows an electron microscope analysis of a purified serotype 1 rAAV/r2c1-GFP virus (x54800).

FIG. 16 shows an electron microscope analysis of a purified serotype 3 rAAV/r2c3-GFP virus (x54800).

FIG. 17 shows an electron microscope analysis of a purified serotype 4 rAAV/r2c4-GFP virus (x54800).

FIG. 18 shows an electron microscope analysis of a purified serotype 5 rAAV/r2c5-GFP virus (x54800).

FIG. 19 shows an electron microscope analysis of a purified serotype 6 rAAV/r2c6-GFP virus (x54800).

FIG. 20 shows an electron microscope analysis of the purified serotype 1 AVV empty capsid virus (x38000).

FIG. 21 shows an electron microscope analysis of the purified serotype 3 AVV empty capsid virus (x38000).

FIG. 22 shows an electron microscope analysis of the purified serotype 4 AVV empty capsid virus (x38000).

FIG. 23 shows an electron microscope analysis of the purified serotype 5 AVV empty capsid virus (x38000).

FIG. 24 shows an electron microscope analysis of the purified serotype 6 AVV empty capsid virus (x38000).

FIG. 25 shows a SDS-PAGE electrophoretogram of the purified serotype 1 rAAV/r2c1-GFP through an ion exchange column during a fine-purification, wherein lane 1: the elution peaks collected by passing rAAV/r2c1 -GFP through an ion exchange column; lane 2: the control of AAV-2; and lane 3: mark;

FIG. 26 shows a SDS-PAGE electrophoretogram of the purified serotype 1 rAAV/r2c1-GFP through a molecular sieve column during a fine-purification, wherein lane 1: mark; lane 2: the elution peaks collected by passing rAAV/r2c1-GFP through a molecular sieve column; and lane 3: the control of AAV-2.

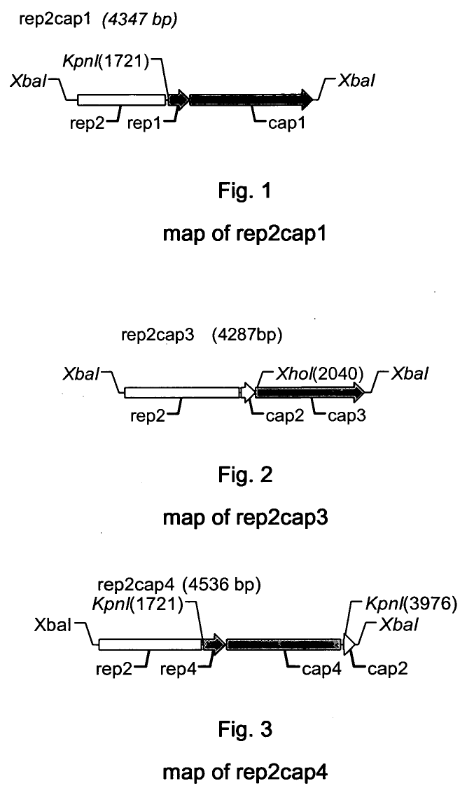

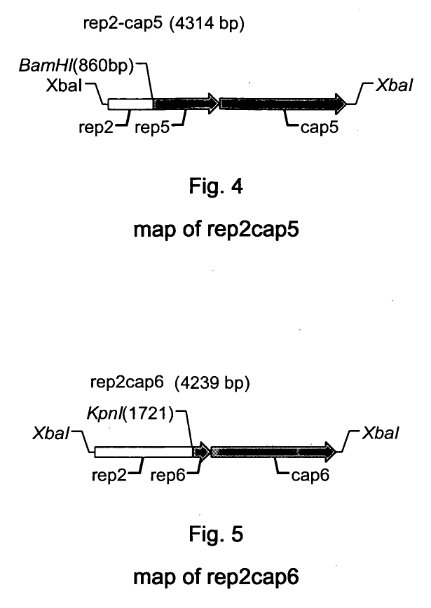

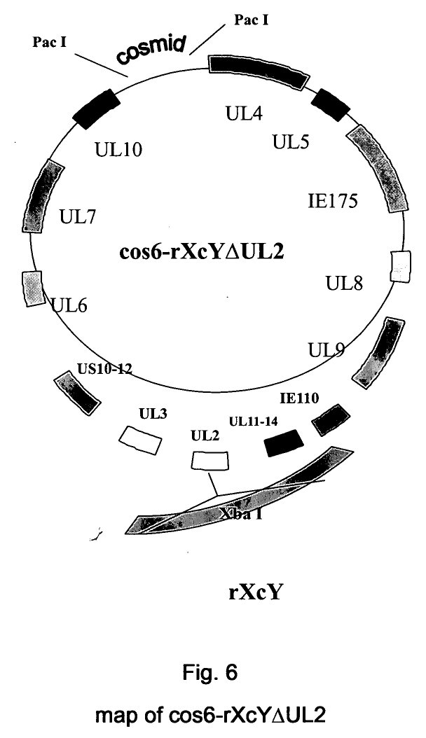

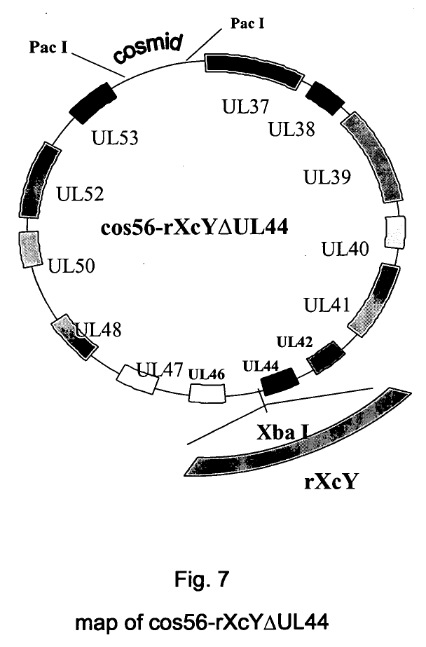

### SPECIFIC EMBODIMENT

Methods for large-scale production, isolation, purification of multi-serotype recombinant adeno-associated virus vectors of the present invention and uses thereof will be described in detail by way of examples with reference to the figures. However, the examples should not be considered as limiting the scope of the present invention.

### EXAMPLE 1

### Method for Large-scale Production, Isolation, Purification of Recombinant Adeno-Associated Virus Serotype 1 Vectors and Uses Thereof

### EXAMPLE 1-1

### Construction of cos6-r2c1□UL2

AAV-1 was used as the template, and the corresponding cap1 (AAV-1) was amplified using PCR method (for primers refer to Primer Sequences 1 and 2). The reaction conditions were 30 circles of 94° for 30 sec, 55° for 30 sec and 72° for 3 min. A PCR segment of 2210 bp, cap1 was obtained. After double digesting with restrictive enzymes KpnI+XbaI, cap1 was ligated with rep 2 of 1721 bp, which was cut from pSSV9 with KpnI+XbaI. The ligation product was inserted into XbaI site of plasmid pGEM-p3zf(+) (Promega Co.) to form p3zf-r2c1 plasmid. After digested from p3zf-r2c1 plasmid with XbaI, r2c1 (of around 4347 bp) was inserted into XbaI site of cos6. As a result, cos6-r2c1□UL2 was obtained.

Primer Sequence 1: Upstream primer of AAV-1 cap: 5′-GTCTGGAGCATGACTTTGGC-3′ (SEQ ID NO: 6);

Primer Sequence 1: Downstream primer of AAV-1 cap:

| | |
|---|---|
| 5′-TCTAGAAGCGCAACCAAGCAGTTAAT-3′. | (SEQ ID NO: 7) |

### EXAMPLE 1-2

### Preparation of Recombinant HSV1-r2c1

Cos6-r2c1□UL2 was mixed at equal molar ratio with five cosmids such as cos14, cos28, cos48, cos56 and the like. Then, the cos framework was removed by PacI enzyme (no need of remove the cos framework by isolation). After that, the mixture was extracted with phenol, phenol/chloroform (1:1) and chloroform respectively. The supernatant was removed, and DNA was precipitated using 2.5 times anhydrous ethanol. 20 ul of lipofactamine (GIBCO BRL) and 10 ug of DNA were used to co-transfect an 80% confluent BHK-21 cell (about 2×10⁶) according to the instructions, then homologous recombination of the 5 HSV-1 segments occurred inside the cells and accordingly HSV1-r2c1 recombinant viruses were generated, respectively. After 24 h of transfection, the cell was cultured in 1640 medium containing 2% FBS at 37°. The medium was changed every day. After 5 days, the cell began exhibit pathology. The supernatant of the medium was collected after complete pathology. The supernatant was centrifugated at 2000 r/min for 5 min. Then, the supernatant was aliquoted and stored at −20°. Thus obtained recombinant viruses were plaque-purified twice so that pure HSV1-r2c1 recombinant viruses were prepared.

### EXAMPLE 1-3

### Construction of AAV Packaging Cell Strains BHK/pSNAV-GFP and BHK/pSNAV-N1-GFP

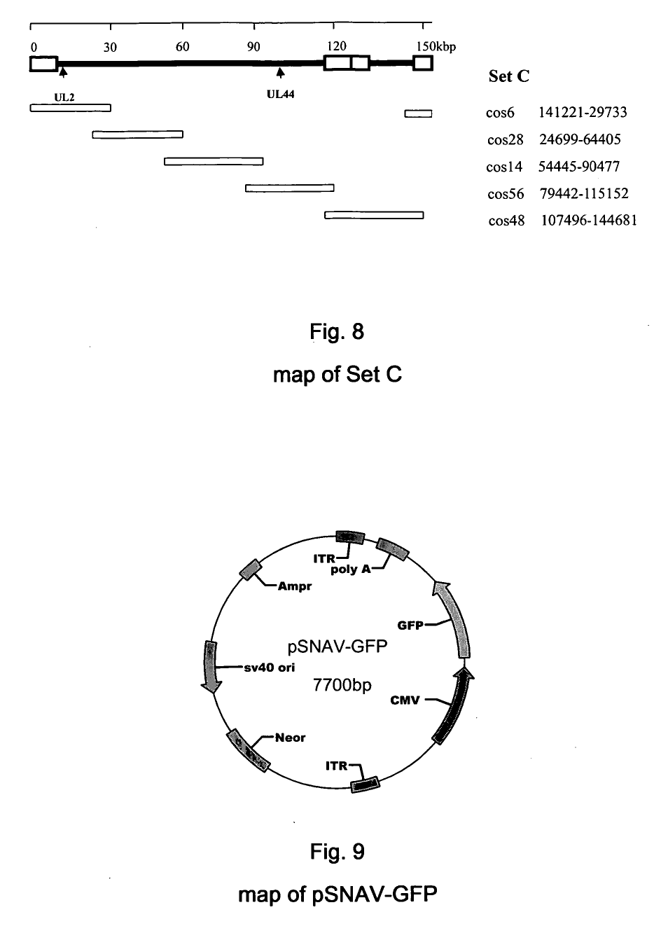

A GFP gene-containing recombinant plasmid pSNAV-GFP was constructed on the basis of the pSNAV-1 plasmid (Wu Zhijian, Wu Xiaobing, Hou Yunde, Construction of a series of adeno-associated virus vectors and study on β-galactosidase expression, Chinese Journal of Virology, 2000,16 (1), 1-6), which is the plasmid comprising “ITR (AAV-2)-exogenous gene-ITR (AAV-2)” and a resistance gene neo^{r} (see FIG. 8). The recombinant plasmid was introduced into BHK-21 cells (ATCC CCL-10) using Liposome Method, the resulting cells were selectively cultured by G418 of 200 ug/ml for 10-15 days. Finally, the obtained resistance cell line was named as BHK/pSNAV-GFP.

AAV-1 and adenovirus 5 were used to infect 293 cells. The cell was freezed and thawed 3 days later, and then was centrifugated (5800 g) for 30 min, and purified with CsCl as described in J.V.1997, 71: 8429-8436. The above-mentioned AAV-1 viruses were treated with 0.1% SDS and protease k of 0.2 mg/ml at 37° for 3 h, then was extracted with phenol/chloroform twice and extracted with chloroform once. Sodium acetate and alcohol were added to precipitate DNA. After deposition, the DNA was resuspensed by TE(PH8.0)at 95° for 5 min and then treated in 0.3-1.0 M NaCl at 50-60° for 2 h until the double-strands was annealed. Qiaex IIgel extraction kit (Qiagen) was used to purify the AAV-1 DNA band of about 5 kb running on agarose gel, then the ends of the DNA band were blunted with Klenow large segment, following addition of a linker of XbaI Linker (dCTCTAGAG) and purification, it was cut with XbaI, loaded in XbaI site of pGEM-3zf (a product of Promega Co.) and amplified in E. Coli DH5α Max Efficiency. After the single clones were picked out and the plasmid was extracted, those clones containing complete AAV-1 genomes were screened via restrictive enzyme and the rep2 probe method. Then, the plasmid was used to transfect BHK cells, and 24 h later the cells were infected by HSV-1. 2 days later, small molecular weight DNAs outside the cell chromosome were extracted using Hirt method, cut with DpnI enzyme, transferred by Southern blotting and hybridized using a rep probe, and the integrity of genomes was indicated by a band of Dimer using a monomer. After all those, pAAV1 was obtained. pAAV1 was double digested with Eco47-3 and NcoI to recove the vector plasmid segment containing AAV-1 ITRs, which was blunted with T4 DNA polymerase; the resistance gene neo^{r} was cut with Bgl II and SmaI from pSV2neo of Promega Co., recovered and blunted with T4 DNA polymerase, and then loaded in the vector plasmid segment containing AAV-1 ITRs. Then, pSNAV-1 was cut with XhoI and BamHII enzyme to recover the CMV-PolyA segment, which was blunted with T4 DNA polymerase, and loaded in the vector plasmid segment containing AAV-1 ITRs. Accordingly, a recombinant plasmid pSNAV-N1 containing AAV-1 ITR elements was obtained.

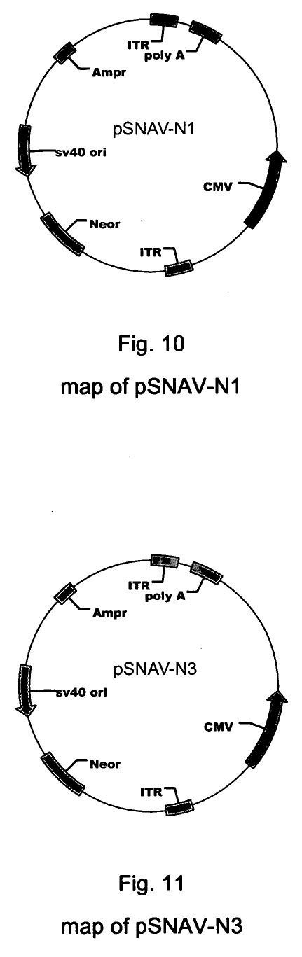

A GFP gene-containing recombinant plasmid pSNAV-N1-GFP was constructed on the basis of the pSNAV-N1 plasmid, wherein the recombinant plasmid pSNAV-N1-GFP contained “ITR (AAV-1)-exogenous gene-ITR (AAV-1)” and a resistance gene neo^{r} (see FIG. 10). The recombinant plasmid was introduced into BHK-21 cells (ATCC CCL-10) using Liposome Method, and then was selectively cultured in G418 at 200 ug/ml for 10-15 days. Finally, the obtained resistance cell line was named as BHK/pSNAV-N1-GFP.

### EXAMPLE 1-4

### Preparation of rAAV/r2c1-GFP having the Serotype of AAV-1

BHK/pSNAV-GFP cells were infected with HSV1-r2c1. After cells exhibited pathology (36-72 h), the cell was repeatedly frozen and thawed for 4 times. Thus, the cell lysis solution contained rAAV/r2c1-GFP and helper viruses HSV1-r2c1. A low-speed centrifugation was carried out to remove the cell debris, and then the lysis solution was treated at 56° for 30 min so that the helper virus HSV1-r2c1 was deactivated. Accordingly, rAAV/r2c1-GFP having the serotype of AAV-1, which could be used to infect a cultured mammal cell in vitro or in vivo, was obtained from the supernatant of the cell lysis solution.

### EXAMPLE 1-5

### Transduction of Cultured Cells in Vitro by rAAV/r2c1-GFP

rAAV/r2c1-GFP viruses (MOI=1) were added to cultured BHK-21 cells (80% confluent). After 24-48 h, a great many green cells could be observed under a fluorescence microscope (excitation wavelength was 490 nm). That shows the resulting rAAV/r2c1-GFP virus is not only infectious but also capable of delivering an exogenous gene into a cell for expression.

### EXAMPLE 1-6

### Production of rAAV/r2c1-GFP Virus having the Serotype of AAV-1 Using Roller Bottle

pSNAV-GFP were introduced into BHK-21 cells (purchased from ATCC and cultured in RPMI1640 medium containing 10% FBS at 37°) using transfecting agent Lipofectamine (produced by GIBCO BRL Co), and then the cells were selectively cultured for 10-15 days in medium with G418 of 800 ug/ml. As a result, vector cells BHK/pSNAV-GFP for mixing cell clones were obtained. Then, after being enlarged propagation, the vector cells were cultured in four 35 cm²-square glass culture vessels; after confluent (about 8×10⁷ cells), the vector cells were digested with trypsin, inoculated into roller bottles (110×288 mm) followed by cultivation under a low-speed (1 rpm) rolling at 37°. The volume of the medium was 200 ml per roller bottle. 3 days later, the cells in the roller bottle were digested with trypsin and then introduced into 5 roller bottles for enlarged cultivation. After confluent (about 2×10⁹ cells), the medium was poured out and 5-10 ml of helper viruses HSV1-r2c1 (MOI=0.5-2) was added in prior to the 1-2 hr of adsorption of the viruses at a low speed (1 rpm). Then, a serum-free 1640 medium (200 ml per roller bottle) was added in to culture the cells at 37° at a low speed (1 rpm). After pathology complete, cells could be shedding easily, the bottle tops were closed tightly before being violently vortexed until all the cells attached to the bottle side were eluted into the medium. After that, the cultures in the 5 roller bottles were pooled and gathered, and after the estimation of their volumes, they were divided into erlenmeyer flasks (specification: 500 ml), 250 ml per flask, for the next purification step.

### EXAMPLE 1-7

### Purification of rAAV/r2c1-GFP Virus having the Serotype of AAV-1

Continuing the above Example. 25 ml of chloroform (10:1 v/v) was added to each erlenmeyer flask, which was placed in a shaker at 37° for 1˜1.5 hr under violent shaking before being taken out and sit at a room temperature for 10 min. DNase and RNase were then added in until the final concentration was 1 μg/ml. Then, after the mixture was blended gently until it became homogeneous, it was being digested at a room temperature for 30-60 min. Solid sodium chloride was added to a final concentration of 1 mol/L under shaking for dissolution. After that, the mixture was centrifugated at 12000 rpm at 4° for 15 min before the upper water phase was extracted and the chloroform and deposits were discarded. Subsequently, PEG8000 was added in until the final concentration was 10% (w/v), and shaken to be dissolved. Then, the mixture was sit at 4° overnight before being centrifugated at 11000 rpm at 4° for 15 min. The supernatant was poured into a clean container (centrifuge tubes were turned upside down on the absorbent paper so that the supernatant could be drained to the full). After that, 5 ml PBS⁺ buffer was used to beat upon, elute and gather the deposits attached to the tube bottoms and tube sides of all the centrifuge tubes. Then the gathered deposits were divided into plastic centrifuge tubes (specification: 1.5 ml), 0.6 ml per tube, before being extracted by chloroform in an equal volume. The extracted mixture were centrifugated at 12000 rpm at 4° for 5 min before the upper water phase was carefully extracted under antiseptic operations and then transferred into an antiseptic tube. The obtained liquid was just a condensed and purified rAAV/r2c1-GFP viral solution, of which the volume was 200 times condensed than its initial volume.

### EXAMPLE 1-8

### Electron Microscope Analysis of rAAV/r2c1-GFP Virus having the Serotype of AAV-1

Solid viral particles with uniform and identical sizes (the particle sizes were around 20-24 mm) could be observed under an electron microscope after the purified rAAV/r2c1-GFP virus solution obtained in the above example was subjected to negative-staining. Please see FIG. 15 in the specification to see the detailed electron microscope results.

### EXAMPLE 1-9

### Titer Determination of rAAV/r2c1-GFP Virus having the Serotype of AAV-1

Continuing Example 1-7. The titer (particles/ml) of the rAAV/r2c1-GFP viruses in the purified viral solution was detected by Dot-Blotting Method using the Digoxigenin-Labeled (Boehringer Mannhein kit) GFP probe. 10 ul of the purified viral solution was diluted 1:10 with PBS²⁺ buffer. DNase and RNase were added in until the final concentration was 1 μg/ml. After the mixture was being digested at 37° for 1 hr and treated in boiling water-bath for 5 min, it was placed in ice bath and then diluted 1:10 with a dilution buffer before dot-blotting (1 ul/dot). Later, the membrane was baked at 120° C. for 30 min, the prehybridization was conducted at 68° C. for 1 hr followed by probe dot-blotting at 68° C. overnight. Afterwards, the membrane was washed and developed. Results: dots 1-4 were definitely positive while dot 5 was weak positive. Supposing the sensibility of detection of DNA by Dot-Blotting Method was 106 molecules, it could be calculated that the viral titer=10⁴⁻⁵×10⁶×10×1000=10¹⁴⁻¹⁵ particles/ml.

### EXAMPLE 1-10

### Infectious Titer Determination of rAAV/r2c1-GFP Virus having the Serotype of AAV

At 37°, a 10% FBS-containing RPMI1640 medium and 5% CO₂ were used to culture HeLa cells, which were then seeded in a 24-well plate (5×10⁵ cell/well). After cultivation overnight, the medium was aspirated; 10 ul of purified rAAV/r2c1-GFP viral solution was diluted 1:10 to 1 ml, then each well was added with 0.5 ml of viral solution with different dilutions and was cultured at 37° for 1 hr; subsequently, each well was added with 50 ul of Ad-5 (MOI=5) and 0.5 ml of medium. After 36 hr of cultivation at 37°, green fluorescent cells could be observed under an inverted fluorescence microscope. After the number of green cells (represented by “n”, wherein 10<n<100) in a certain well had been counted, the viral titer of rAAV/r2c1-GFP could be calculated as follows: n×Times of Dilution×1000/5=n×10⁹×200=2n×10¹¹ TU/ml. Thus, it could be estimated that the infectious titer of the rAAV/r2c1-GFP virus was between 2×10¹² TU/ml and 2×10¹³ TU/ml.

### EXAMPLE 1-11

### Production and Purification of AVV Empty Capsid Virion

BHK-21 cells were cultured in roller bottles. After cells were confluent, helper viruses HSV1-r2c1 were added and pathological cell culture was obtained using the same process according to Example 1-6, and the AVV virus of the culture was extracted using the rAVV purification method according to the present invention. By observing the obtained viral solution under an electron microscope (Please see FIG. 20 in the specification), a great many viral particles with a high core density could be visualized. That showed the observed viral particles were empty capsids. The result shows that by using helper viruses HSV1-r2c1 to infect the BHK cell which does not be transfected by AVV vector DNA (ITR sequence not included), empty capsid of AVV viral can be produced effectively.

### EXAMPLE 1-12

### Futher Purification of rAAV/r2c1-GFP Virus having the Serotype of AAV1

Continuing Example 1-7. A product in a purity of above 95% (SDS-PAGE), with a titer of 7×10¹¹ vg/ml, with more than 20% expression at an in vitro expression MOI value of 1×10⁵ and with an acceptable amount of residual nucleic acid, could be obtained from a crudely purified AAV-1 rAAV/r2c1-GFP virus sample via column purification. The obtained rAAV/r2c1-GFP viral solution sample was loaded to a QFF column which had been balanced with a buffer (0.1×PBS; PH=8.0) at a loading flow rate of 5-10 ml/min. Then, the QFF column was balanced with a buffer (0.1×PBS; PH=8.0) once again; later, after the column was eluted by a buffer (0.1×PBS; 1M NaCl; PH=8.0), the elution peaks with conductance values from 9.0 to 21.0 ms/cm were collected.

The collected elution peak samples were loaded to a S200 column which had been balanced with a buffer (1×PBS; PH=7.4) at a loading flow rate of 1.5-5 ml/min. Then, after the column was eluted by a buffer (1×PBS; PH=7.4), the elution peaks were collected so that a further purified clinical-grade rAAV was obtained.

### EXAMPLE 2

### Method for Large-scale Production, Isolation, Purification Recombinant Adeno-Associated Virus Serotype 3 Vectors of and Uses thereof

### EXAMPLE 2-1

### Construction of cos6-r2c3□UL2

AAV-3 was used as the template, and the corresponding cap3 (AAV-3) was amplified using PCR method (primers refer to Primer Sequences 3 and 4). The reaction conditions were 30 circles of 94° for 30 sec, 55° for 30 sec and 72° for 3 min. A PCR segment of 2040 bp, cap3 was obtained. After double digesting with restrictive enzymes XhoI and XbaI, cap3 was ligated with rep 2 of 2040 bp, which was digested from pSSV9 with XhoI and XbaI. The ligation product was inserted into XbaI site of plasmid pGEM-p3zf(+) (Promega Co.) to form p3zf-r2c3 plasmid. After cut from p3zf-r2c3 plasmid with XbaI, r2c3 (of around 4287 bp) was inserted into XbaI site of cos6. As a result, cos6-r2c3□UL2 was obtained.

Primer Sequence 3: Upstream primer of AAV-3 cap:

| | |
|---|---|
| 5′-TCTAGAGGTCAAAGAGACTGTGGGGA-3′; | (SEQ ID NO: 8) |

Primer Sequence 1: Downstream primer of AAV-3 cap:

| | |
|---|---|
| 5′-TCTAGATGCACAAGAGCCAAAGTTCA-3′. | (SEQ ID NO: 9) |

### EXAMPLE 2-2

### Preparation of Recombinant HSV1-r2c3

Cos6-r2c3□UL2 was mixed at equal molar ratio with five cosmids such as cos14, cos28, cos48, cos56 and the like. Then, the cos framework was removed by PacI enzyme (no need of remove the cos framework by isolation). After that, the mixture was extracted with phenol, phenol/chloroform (1:1) and chloroform, respectively. The supernatant was aspirated and collected, and DNA was precipitated using 2.5 times anhydrous ethanol. 20 ul of lipofactamine (GIBCO BRL) and 10 ul of DNA were used to co-transfect 80% confluent BHK-21 cells (about 2×10⁶ cells) according to the instructions, then homologous recombination of the 5 HSV-1 segments occurred inside the cells and accordingly HSV1-r2c3 recombinant viruses were generated, respectively. After 24 h of transfection, the cell was cultured in 1640 medium containing 2% FBS at 37°. The medium was changed every day. After 5 days, cells began to exhibit pathology. The supernatant of the medium was collected after complete pathology followed by centrifugating at 2000 r/min for 5 min. Then, the supernatant was aliquoted and stored at −20°. The obtained recombinant viruses were plaque-purified twice so that pure HSV1-r2c3 recombinant viruses were prepared.

### EXAMPLE 2-3

### Construction of AAV Packaging Cell Strain BHK/pSNAV-N3-GFP

AAV-3 and adenovirus 1 were used to infect 293 cells. 3 days later, the cells were frozen and thawed, and then were centrifugated (5800 g) for 30 min, and purified with CsCl as described in J.V.1997, 71: 8429-8436. The above-mentioned AAV-3 viruses were treated with 0.1% SDS and protease k of 0.2 mg/ml at 37° for 3 h, then was extracted with phenol/chloroform twice and extracted with chloroform once. Sodium acetate and alcohol were added to precipitate DNA. After deposition, the DNA was resuspensed by TE(PH8.0)at 95° for 5 min and then treated in 0.3-1.0 M NaCl at 50-60° for 2 h until the double-strands were annealed. Qiaex IIgel extraction kit (Qiagen) was used to purify the AAV-3 DNA band of about 5 kb running out from agarose gel, then the ends of the DNA band were blunted with Klenow large segment, and after addition of a linker of XbaI Linker (dCTCTAGAG) and purification, it was cut with XbaI, loaded in XbaI site of pGEM-3zf (a product of Promega Co.) and amplified in E. Coli DH5α Max Efficiency. After the single clones were picked out and the plasmid was extracted, those clones containing complete AAV-3 genomes were screened via restrictive enzyme and the rep2 probe method. Then, the plasmid was used to transfect BHK cells and 24 h later the cells were infected by HSV-1. 2 days later, small molecular weight DNAs outside the cell chromosome were extracted using Hirt method, cut with DpnI enzyme, transferred by Southern blotting and hybridized using a rep probe, and the integrity of genomes was verified by Dimer band using a monomer. After all those, pAAV3 was obtained. The pAAV3 was cut with BssHI and ApalI enzymes to recover the vector plasmid segment containing AAV-3 ITRs, then blunted with T4 DNA polymerase. Then, pSNAV-1 was cut with XhoI and BamHI enzymes to recover the CMV-PolyA segment, then blunted with T4 DNA polymerase, and was then loaded in the vector plasmid segment containing AAV-3 ITRs, the resistance gene neo^{r} was cut with Bgl II and Smal enzymes from pSV2neo of Promega Co., followed by blunting with T4 DNA polymerase and recovered, and then loaded in the vector plasmid segment containing AAV-3 ITRs. Accordingly, a recombinant plasmid pSNAV-N3 containing AAV-3 ITR elements was obtained.

A GFP gene-containing recombinant plasmid pSNAV-N3-GFP was constructed on the basis of the pSNAV-N3 plasmid, wherein the recombinant plasmid pSNAV-N3-GFP contained “ITR (AAV-3)-exogenous gene -ITR (AAV-3)” and a resistance gene neo^{r} (please see FIG. 11). The recombinant plasmid was introduced into BHK-21 cells (ATCC CCL-10) using Liposome Method, then was selectively cultured in G418 of 200 ug/ml for 10-15 days. Finally, the obtained resistance cell line was named as BHK/pSNAV-N3-GFP.

### EXAMPLE 2-4

### Preparation of rAAV/r2c3-GFP having the Serotype of AAV-3

BHK/pSNAV-GFP cells were infected with HSV1-r2c3. After cells exhibited pathology (36-72 h), the cell was repeatedly frozen and thawed for 4 times. Thus, the cell lysis solution contained rAAV/r2c3-GFP and helper viruses HSV1-r2c3. A low-speed centrifugation was carried out to remove the cell debris, and then the lysis solution was treated at 56° for 30 min so that the helper virus HSV1-r2c3 was deactivated. Accordingly, rAAV/r2c3-GFP having the serotype of AAV-3, which could be used to infect a cultured mammal cell in vitro and in vivo, was obtained from the supernatant of the cell lysis solution.

### EXAMPLE 2-5

### Transduction of Cultured Cells In Vitro by rAAV/r2c3-GFP

rAAV/r2c3-GFP viruses (MOI=1) were added to cultured BHK-21 cells (80% confluent). After 24-48 h, a great many green cells could be observed under a fluorescence microscope (excitation wavelength was 490 nm). That shows the resulting rAAV/r2c3-GFP virus is not only infectious but also capable of delivering an exogenous gene into a cell for expression.

### EXAMPLE 2-6

### Production of rAAV/r2c1-GFP Viruses having the Serotype of AAV-3 Using Roller Bottle

pSNAV-GFPs were introduced into BHK-21 cells (purchased from ATCC and cultured in RPMI1640 medium containing 10% FBS at 37°) using transfecting agent Lipofectamine (produced by GIBCO BRL Co), and then the cells were selectively cultured for 10-15 days in medium with G418 of 800 ug/ml. As a result, vector cells BHK/pSNAV-GFP for mixing cell clones were obtained. Then, after being enlarged propagation, the vector cells were cultured in four 35 cm²-square glass culture vessels; after confluent (about 8×10⁷ cells), the vector cells were digested with trypsin, inoculated into roller bottles (110×288 mm) followed by cultivation under a low-speed (1 rpm) rolling at 37°. The volume of the medium was 200 ml per roller bottle. 3 days later, the cells in the roller bottle were digested with trypsin and then introduced into 5 roller bottles for enlarged cultivation. After confluent (about 2×10⁹ cells), the medium was poured out and 5-10 ml of helper viruses HSV1-r2c3(MOI=0.5-2) was added in prior to the 1-2 hr of adsorption of the viruses at a low speed (1 rpm). Then, a serum-free 1640 medium (200 ml per roller bottle) was added in to culture the cells at 37° at a low speed (1 rpm). After pathology complete, cells could be shedding easily, the bottle tops were closed tightly before being violently vortexed until all the cells attached to the bottle side were eluted into the medium. After that, the cultures in the 5 roller bottles were pooled and gathered, and after the estimation of their volumes, they were divided into erlenmeyer flasks (specification: 500 ml), 250 ml per flask, for the next purification step.

### EXAMPLE 2-7

### Purification of rAAV/r2c3-GFP Virus having the Serotype of AAV-3

Continuing the above Example. 25 ml of chloroform (10:1 v/v) was added to each erlenmeyer flask, which was placed in a shaker at 37° for 1˜1.5 hr under violent shaking before being taken out and sit at a room temperature for 10 min. DNase and RNase were then added in until the final concentration was 1 μg/ml. Then, after the mixture was blended gently until it became homogeneous, it was being digested at a room temperature for 30-60 min. Solid sodium chloride was added to a final concentration of 1 mol/L under shaking for dissolution. After that, the mixture was centrifugated at 12000 rpm at 4° for 15 min before the upper water phase was aspirated and the chloroform and deposits were discarded. Subsequently, PEG8000 was added in until the final concentration was 10% (w/v), and shaken to be dissolved. Then, the mixture was sit at 4° overnight before being centrifugated at 11000 rpm at 4° for 15 min. The supernatant was poured into a clean container (centrifuge tubes were turned upside down on the absorbent paper so that the supernatant could be drained to the full). After that, 5 ml PBS⁺ buffer was used to beat upon, elute and gather the deposits attached to the tube bottoms and tube sides of all the centrifuge tubes. Then the gathered deposits were divided into plastic centrifuge tubes (specification: 1.5 ml), 0.6 ml per tube, before being extracted by chloroform in an equal volume. The extracted mixture were centrifugated at 12000 rpm at 4° for 5 min before the upper water phase was carefully aspirated under antiseptic operations and then transferred into an antiseptic tube. The obtained liquid was just a condensed and purified rAAV/r2c3-GFP viral solution, of which the volume was 200 times condensed than its initial volume.

### EXAMPLE 2-8

### Electron Microscope Analysis of rAAV/r2c3-GFP Virus having the Serotype of AAV-3

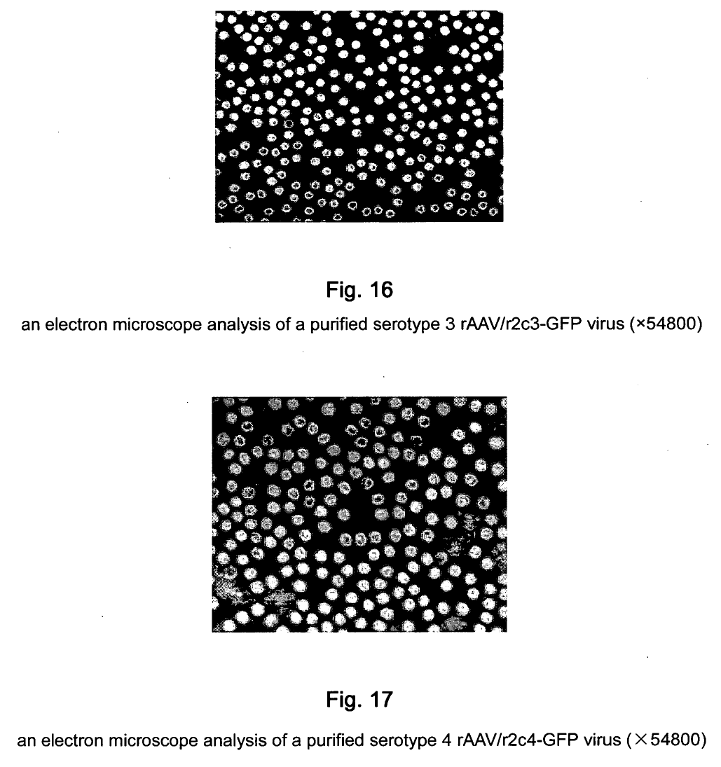

Solid viral particles with uniform and identical sizes (the particle sizes were around 20-24 mm) could be observed under an electron microscope after the purified rAAV/r2c3-GFP virus solution obtained in the above example was subjected to negative-staining. Please see FIG. 16 in the specification to see the detailed electron microscope results.

### EXAMPLE 2-9

### Titer Determination of rAAV/r2c3-GFP Virus having the Serotype of AAV-3

Continuing Example 2-7. The titer (particles/ml) of the rAAV/r2c3-GFP viruses in the purified viral solution was detected by Dot-Blotting Method using the Digoxigenin-Labeled (Boehringer Mannhein kit) GFP probe. 10 ul of the purified viral solution was diluted 1:10 with PBS²⁺ buffer. DNase and RNase were added in until the final concentration was 1 μg/ml. After the mixture was being digested at 37° for 1 hr and treated in boiling water-bath for 5 min, it was placed in ice bath and then diluted 1:10 with a dilution buffer before dot-blotting (1 ul/dot). Later, the membrane was baked at 120° C. for 30 min, the prehybridization was conducted at 68° C. for 1 hr followed by probe dot-blotting at 68° C. overnight. Afterwards, the membrane was washed and developed. Results: dots 1-4 were definitely positive while dot 5 was weak positive. Supposing the sensibility of detection of DNA by Dot-Blotting Method was 10⁶ molecules, it could be calculated that the viral titer=10⁴⁻⁵×10⁶×10×1000=10¹⁴⁻¹⁵ particles/ml.

### EXAMPLE 2-10

### Infectious Titer Determination of rAAV/r2c3-GFP Virus having the Serotype of AAV-3

At 37°, a 10% FBS-containing RPMI1640 medium and 5% CO₂ were used to culture HeLa cells, which were then seeded in a 24-well plate (5×10⁵ cell/well). After cultivation overnight, the medium was aspirated; 10 ul of purified rAAV/r2c1-GFP viral solution was diluted 1:10 to 1 ml, then each well was added with 0.5 ml of viral solution with different dilutions and was cultured at 37° for 1 hr; subsequently, each well was added with 50 ul of Ad-5 (MOI=5) and 0.5 ml of medium. After 36 hr of cultivation at 37°, green fluorescent cells could be observed under an inverted fluorescence microscope. After the number of green cells (represented by “n”, wherein 10<n<100) in a certain well had been counted, the viral titer of rAAV/r2c3-GFP could be calculated as follows: n×Times of Dilution×1000/5=n×10⁹×200=2n×10¹¹ TU/ml. Thus, it could be estimated that the infectious titer of the rAAV/r2c3-GFP virus was between 2×10¹² TU/ml and 2×10¹³ TU/ml.

### EXAMPLE 2-11

### Production and Purification of AVV Empty Capsid Virion

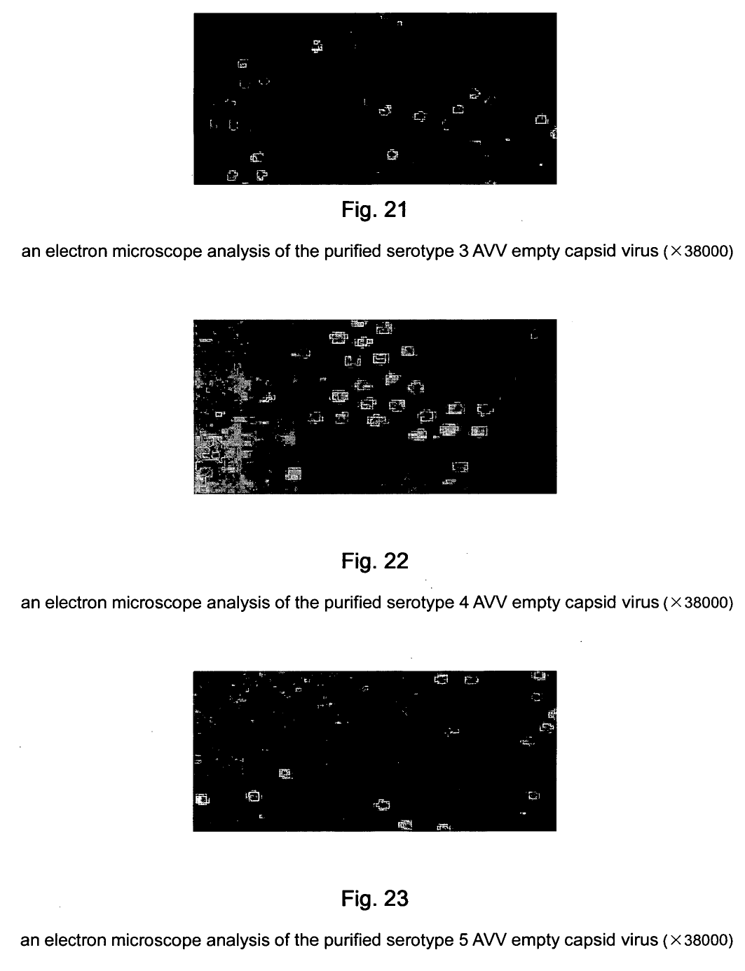

BHK-21 cells were cultured in roller bottles. After cells were confluent, helper viruses HSV1-r2c3 were added and pathological cell culture was obtained using the same process according to Example 2-6, and the AVV virus of the culture was extracted using the rAVV purification method according to the present invention. By observing the obtained viral solution under an electron microscope (Please see FIG. 21 in the specification), a great many viral particles with a high core density could be visualized. That showed the observed viral particles were empty capsids. The result shows that by using helper viruses HSV1-r2c3 to infect the BHK cell which does not be transfected by AVV vector DNA (ITR sequence not included), empty capsid of AVV viral can be produced effectively.

### EXAMPLE 3

### Method for Large-scale Production, Isolation, Purification of Recombinant Adeno-Associated Virus Serotype 4 Vectors and Uses Thereof

### EXAMPLE 3-1

### Construction of cos6-r2c4□UL2

AAV-1 was used as the template, and the corresponding cap4 (AAV-4) was amplified using PCR method (primers refer to Primer Sequences 5 and 6). The reaction conditions were 30 circles of 94° for 30 sec, 55° for 30 sec and 72° for 3 min. A PCR segment of 2255 bp, cap4 was obtained. After digesting with restrictive enzyme KpnI, cap4 was linked with a large segment to form a SSV9-cap4 plasmid, which segment was cut from pSSV9 with KpnI without the DNA segment of cap2. After digested from SSV9-cap4 plasmid with XbaI, r2c4 (of around 4536 bp) was inserted into XbaI site of cos6. As a result, cos6-r2c4□UL2 was obtained.

Primer Sequence 5: Upstream primer of AAV-4 cap: 5′-GCGGACAGGTACCAAAACAA-3′ (SEQ ID NO: 10);

Primer Sequence 6: Downstream primer of AAV-4 cap:

| | |
|---|---|
| 5′-GAAGGATTCGCAGGTACCGG-3′. | (SEQ ID NO: 11) |

### EXAMPLE 3-2

### Preparation of Recombinant HSV1-r2c4

Cos6-r2c4□UL2 was mixed at equal molar ratio with five cosmids such as cos14, cos28, cos48, cos56 and the like. Then, the cos framework was removed by PacI enzyme (no need of remove the cos framework by isolation). After that, the mixture was extracted with phenol, phenol/chloroform (1:1) and chloroform, respectively. The supernatant was aspirated and collected, and DNA was precipitated using 2.5 times anhydrous ethanol. 20 ul of lipofactamine (GIBCO BRL) and 10 ul of DNA were used to co-transfect 80% confluent BHK-21 cells (about 2×10⁶ cells) according to the instructions, then homologous recombination of the 5 HSV-1 segments occurred inside the cells and accordingly HSV1-r2c4 recombinant viruses were generated, respectively. After 24 h of transfection, the cell was cultured in 1640 medium containing 2% FBS at 37°. The medium was changed every day. After 5 days, cells began to exhibit pathology. The supernatant of the medium was collected after complete pathology followed by centrifugating at 2000 r/min for 5 min. Then, the supernatant was aliquoted and stored at −20°. The obtained recombinant viruses were plaque-purified twice so that pure HSV1-r2c4 recombinant viruses were prepared.

### EXAMPLE 3-3

### Construction of AAV Packaging Cell Strain BHK/pSNAV-N4-GFP

AAV-4 and adenovirus 5 were used to infect 293 cells. 3 days later, the cells were frozen and thawed, and then were centrifugated (5800 g) for 30 min, and purified with CsCl as described in J.V.1997, 71: 8429-8436. The above-mentioned AAV-3 viruses were treated with 0.1% SDS and protease k of 0.2 mg/ml at 37° for 3 h, then was extracted with phenol/chloroform twice and extracted with chloroform once. Sodium acetate and alcohol were added to precipitate DNA. After deposition, the DNA was resuspensed by TE(PH8.0)at 95° for 5 min and then treated in 0.3-1.0 M NaCl at 50-60° for 2 h until the double-strands was annealed. Qiaex IIgel extraction kit(Qiagen) was used to purify the AAV-3 DNA band of about 5 kb running out from agarose gel, then the ends of the DNA band were blunted with Klenow large segment, and after addition of a linker of XbaI Linker (dCTCTAGAG) and purified, it was cut with XbaI, loaded in XbaI site of pGEM-3zf (a product of Promega Co.) and amplified in E. Coli DH5α Max Efficiency. After the single clones were picked out and the plasmid was extracted, those clones containing complete AAV-4 genomes were screened via restrictive enzyme and the rep2 probe method. Then, the plasmid was used to transfect BHK cells and 24 h later the cells were infected by HSV-1. 2 days later, small molecular weight DNAs outside the cell chromosome were extracted using Hirt method, cut with DpnI enzyme, transferred by Southern blotting and hybridized using a rep probe, and the integrity of genomes was verified by Dimer band using a monomer. After all those, pAAV4 was obtained. pAAV4 was cut with Ava II and NcoI enzymes to recover the vector plasmid segment containing AAV-4 ITRs, then blunted with T4 DNA polymerase; pSNAV-GFP was cut with XhoI and BamHI enzymes to recover the CMV-PolyA segment, then blunted with T4 DNA polymerase, and was then loaded in the vector plasmid segment containing AAV-4 ITRs. Then, the resistance gene neo^{r} from pSV2neo of Promega Co. was cut with Bgl II and Smal enzymes to recover the resistance gene neo^{r} followed by blunting with T4 DNA polymerase, and was then loaded in the vector plasmid segment containing AAV-4 ITRs. Accordingly, a recombinant plasmid pSNAV-N4 containing AAV-4 ITR elements was obtained.

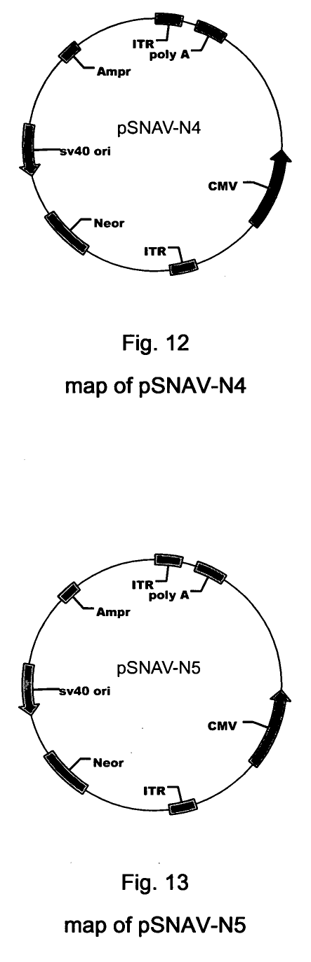

A GFP gene-containing recombinant plasmid pSNAV-N4-GFP was constructed on the basis of the pSNAV-N4 plasmid, wherein the recombinant plasmid pSNAV-N4-GFP contained “ITR (AAV-4)-exogenous gene -ITR (AAV-4)” and a resistance gene neo^{r} (please see FIG. 12). The recombinant plasmid was introduced into BHK-21 cells (ATCC CCL-10) using Liposome Method, then was selectively cultured in G418 of 200 ug/ml for 10-15 days. Finally, the obtained resistance cell line was named as BHK/pSNAV-N4-GFP.

### EXAMPLE 3-4

### Preparation of rAAV/r2c4-GFP having the Serotype of AAV-4

BHK/pSNAV-GFP cells were infected with HSV1-r2c4. After cells exhibited pathology (36-72 h), the cell was repeatedly frozen and thawed for 4 times. Thus, the cell lysis solution contained rAAV/r2c4-GFP and helper viruses HSV1-r2c4. A low-speed centrifugation was carried out to remove the cell debris, and then the lysis solution was treated at 56° for 30 min so that the helper virus HSV1-r2c4 was deactivated. Accordingly, rAAV/r2c4-GFP having the serotype of AAV-4, which could be used to infect a cultured mammal cell in vitro/in vivo, was obtained from the supernatant of the cell lysis solution.

### EXAMPLE 3-5

### Transduction of Cultured Cells In Vitro by rAAV/r2c4-GFP

rAAV/r2c4-GFP viruses (MOI=1) were added to cultured BHK-21 cells (80% confluent). After 24-48 h, a great many green cells could be observed under a fluorescence microscope (excitation wavelength was 490 nm). That shows the resulting rAAV/r2c4-GFP virus is not only infectious but also capable of delivering an exogenous gene into a cell for expression.

### EXAMPLE 3-6

### Production of rAAV/r2c4-GFP Viruses having the Serotype of AAV-4 Using Roller Bottle

pSNAV-GFPs were introduced into BHK-21 cells (purchased from ATCC and cultured in RPMI1640 medium containing 10% FBS at 37°) using transfecting agent Lipofectamine (produced by GIBCO BRL Co), and then the cells were selectively cultured for 10-15 days in medium with G418 of 800 ug/ml. As a result, vector cells BHK/pSNAV-GFP for mixing cell clones were obtained. Then, after being enlarged propagation, the vector cells were cultured in four 35 cm²-square glass culture vessels; after confluent (about 8×10⁷ cells), the vector cells were digested with trypsin, inoculated into roller bottles (110×288 mm) followed by cultivation under a low-speed (1 rpm) rolling at 37°. The volume of the medium was 200 ml per roller bottle. 3 days later, the cells in the roller bottle were digested with trypsin and then introduced into 5 roller bottles for enlarged cultivation. After confluent (about 2×10⁹ cells), the medium was poured out and 5-10 ml of helper viruses HSV1-r2c4(MOI=0.5-2) was added in prior to the 1-2 hr of adsorption of the viruses at a low speed (1 rpm). Then, a serum-free 1640 medium (200 ml per roller bottle) was added in to culture the cells at 37° at a low speed (1 rpm). After pathology complete, cells could be shedding easily, the bottle tops were closed tightly before being violently vortexed until all the cells attached to the bottle side were eluted into the medium by shaken acutely. After that, the cultures in the 5 roller bottles were pooled and gathered, and after the estimation of their volumes, they were divided into erlenmeyer flasks (specification: 500 ml), 250 ml per flask, for the next purification step.

### EXAMPLE 3-7

### Purification of rAAV/r2c4-GFP Virus having the Serotype of AAV-4

Continuing the above Example. 25 ml of chloroform (10:1 v/v) was added to each erlenmeyer flask, which was placed in a shaker at 37° for 1˜1.5 hr under violent shaking before being taken out and sit at a room temperature for 10 min. DNase and RNase were then added in until the final concentration was 1 μg/ml. Then, after the mixture was blended gently until it became homogeneous, it was being digested at a room temperature for 30-60 min. Solid sodium chloride was added to a final concentration of 1 mol/L under shaking for dissolution. After that, the mixture was centrifugated at 12000 rpm at 4° for 15 min before the upper water phase was aspirated and the chloroform and deposits were discarded. Subsequently, PEG8000 was added in until the final concentration was 10% (w/v), and shaken to be dissolved. Then, the mixture was sit at 4° overnight before being centrifugated at 11000 rpm at 4° for 15 min. The supernatant was poured into a clean container (centrifuge tubes were turned upside down on the absorbent paper so that the supernatant could be drained to the full). After that, 5 ml PBS⁺ buffer was used to beat upon, elute and gather the deposits attached to the tube bottoms and tube sides of all the centrifuge tubes. Then the gathered deposits were divided into plastic centrifuge tubes (specification: 1.5 ml), 0.6 ml per tube, before being extracted by chloroform in an equal volume. The extracted mixture were centrifugated at 12000 rpm at 4° for 5 min before the upper water phase was carefully aspirated via an antiseptic operation and then transferred into an antiseptic tube. The obtained liquid was just a condensed and purified rAAV/r2c4-GFP viral solution, of which the volume was 200 times condensed than its initial volume.

### EXAMPLE 3-8

### Electron Microscope Analysis of rAAV/r2c4-GFP Virus having the Serotype of AAV-4

Solid viral particles with uniform and identical sizes (the particle sizes were around 20-24 mm) could be observed under an electron microscope after the purified rAAV/r2c4-GFP virus solution obtained in the above example was subjected to negative-staining. Please see FIG. 17 in the specification to see the detailed electron microscope results.

### EXAMPLE 3-9

### Titer Determination of rAAV/r2c4-GFP Virus Negative-staining

Continuing Example 3-7. The titer (particles/ml) of the rAAV/r2c4-GFP viruses in the purified viral solution was detected by Dot-Blotting Method using the Digoxigenin-Labeled (Boehringer Mannhein kit) GFP probe. 10 ul of the purified viral solution was diluted 1:10 with PBS²⁺ buffer. DNase and RNase were added in until the final concentration was 1 μg/ml. After the mixture was being digested at 37° for 1 hr and treated in boiling water-bath for 5 min, it was placed in ice bath and then diluted 1:10 with a dilution buffer before dot-blotting (1 ul/dot). Later, the membrane was baked at 120° C. for 30 min, the prehybridization was conducted at 68° C. for 1 hr followed by probe dot-blotting at 68° C. overnight. Afterwards, the membrane was washed and developed. Results: dots 1-4 were definitely positive while dot 5 was weak positive. Supposing the sensibility of detection of DNA by Dot-Blotting Method was 10⁶ molecules, it could be calculated that the viral titer=10⁴⁻⁵×10⁶×10×1000=10¹⁴⁻¹⁵ particles/ml.

### EXAMPLE 3-10

### Infectious Titer Determination of rAAV/r2c4-GFP Virus Negative-staining

At 37°, a 10% FBS-containing RPMI1640 medium and 5% CO₂ were used to culture HeLa cells, which were then seeded in a 24-well plate (5×10⁵ cell/well). After cultivation overnight, the medium was absorbed out; 10 ul of purified rAAV/r2c4-GFP viral solution was diluted 1:10 to 1 ml, then each well was added with 0.5 ml of viral solution with different dilutions and was cultured at 37° for 1 hr; subsequently, each well was added with 50 ul of Ad-5(MOI=5) and 0.5 ml of medium. After 36 hr of cultivation at 37°, green fluorescent cells could be observed under an inverted fluorescence microscope. After the number of green cells (represented by “n”, wherein 10<n<100) in a certain well had been counted, the viral titer of rAAV/r2c4-GFP could be calculated as follows: n×Times of Dilution×1000/5=n×10⁹×200=2n×10¹¹ TU/ml. Thus, it could be estimated that the infectious titer of the rAAV/r2c4-GFP virus was between 2×10¹² TU/ml and 2×10¹³ TU/ml.

### EXAMPLE 3-11

### Production and Purification of AVV Empty Capsid Virion

BHK-21 cells were cultured in roller bottles. After cells were confluent, helper viruses HSV1-r2c4 were added and pathological cell culture was obtained using the same process according to Example 3-6, and the AVV virus of the culture was extracted using the rAVV purification method according to the present invention. By observing the obtained viral solution under an electron microscope (Please see FIG. 22 in the specification), a great many viral particles with a high core density could be visualized. That showed the observed viral particles were empty capsids. The result shows that by using helper viruses HSV1-r2c4 to infect the BHK cell which does not be transfected by AVV vector DNA (ITR sequence not included), empty capsid of AVV viral can be produced effectively.

### EXAMPLE 4

### Method for Large-scale Production, Isolation, Purification of Recombinant Adeno-Associated Virus Serotype 5 Vectors and Uses Thereof

### EXAMPLE 4-1

### Construction of cos6-r2c5□UL2

AAV-5 was used as the template, and the corresponding cap5 (AAV-5) was amplified using PCR method (primers refer to Primer Sequences 7 and 8). The reaction conditions were 30 circles of 94° for 30 sec, 55° for 30 sec and 72° for 3 min. A PCR segment of 2170 bp, cap5 was obtained. After double digesting with restrictive enzymes BamHI and XbaI, cap1 was ligated with rep 2 of 860 bp, which was digested from pSSV9 with BamHI and XbaI. The ligation product was inserted into XbaI site of plasmid pGEM-p3zf(+) (Promega Co.) to form p3zf-r2c5 plasmid. After digested from p3zf-r2c5 plasmid with XbaI, r2c5 (of around 4314 bp) was inserted into XbaI site of cos6. As a result, cos6-r2c5□UL2 was obtained.

Primer Sequence 7: Upstream primer of AAV-5 cap: 5′-GGATCCAGGAAAATCAGGAG-3′ (SEQ ID NO: 12);

Primer Sequence 8: Downstream primer of AAV-5 cap:

| | |
|---|---|
| 5′-TCTAGACATGAATGGGTTAAAGGG-3′. | (SEQ ID NO: 13) |

### EXAMPLE 4-2

### Preparation of Recombinant HSV1-r2c5

Cos6-r2c5□UL2 was mixed at equal molar ratio with five cosmids such as cos14, cos28, cos48, cos56 and the like. Then, the cos framework was removed by PacI enzyme (no need of remove the cos framework by isolation). After that, the mixture was extracted with phenol, phenol/chloroform (1:1) and chloroform, respectively. The supernatant was aspirated and collected, and DNA was precipitated using 2.5 times anhydrous ethanol. 20 ul of lipofactamine (GIBCO BRL) and 10 ul of DNA were used to co-transfect 80% confluent BHK-21 cells (about 2×10⁶ cells) according to the instructions, then homologous recombination of the 5 HSV-1 segments occurred inside the cells and accordingly HSV1-r2c5 recombinant viruses were generated, respectively. After 24 h of transfection, the cell was cultured in 1640 medium containing 2% FBS at 37°. The medium was changed every day. After 5 days, cells began to exhibit pathology. The supernatant of the medium was collected after complete pathology followed by centrifugating at 2000 r/min for 5 min. Then, the supernatant was aliquoted and stored at −20°. The obtained recombinant viruses were plaque-purified twice so that pure HSV1-r2c5 recombinant viruses were prepared.

### EXAMPLE 4-3

### Construction of AAV Packaging Cell Strain BHK/pSNAV-N5-GFP

AAV-5 and adenovirus 5 were used to infect 293 cells. 3 days later, the cells were frozen and thawed, and then were centrifugated (5800 g) for 30 min, and purified with CsCl as described in J.V.1997, 71: 8429-8436. The abovementioned AAV-4 viruses were treated with 0.1% SDS and protease k of 0.2 mg/ml at 37° for 3 h, then was extracted with phenol/chloroform twice and extracted with chloroform once. Sodium acetate and alcohol were added to precipitate DNA. After deposition, the DNA was resuspensed by TE(PH8.0)at 95° for 5 min and then treated in 0.3-1.0 M NaCl at 50-60° for 2 h until the double-strands was annealed. Qiaex IIgel extraction kit (Qiagen) was used to purify the AAV-4 DNA band of about 5 kb running out from agarose gel, then the ends of the DNA band were blunted with Klenow large segment, and after addition of a linker of XbaI Linker (dCTCTAGAG) and purified, it was cut with XbaI, loaded in XbaI site of pGEM-3zf (a product of Promega Co.) and amplified in E. Coli DH5α Max Efficiency. After the single clones were picked out and the plasmid was extracted, those clones containing complete AAV-4 genomes were screened via restrictive enzyme and the rep2 probe method. Then, the plasmid was used to transfect BHK cells and 24 h later the cells were transfected by HSV-1. 2 days later, small molecular weight DNAs outside the cell chromosome were extracted using Hirt method, cut with DpnI enzyme, transferred by Southern blotting and hybridized using a rep probe, and the integrity of genomes was verified by Dimer band using a monomer. After all those, pAAV5 was obtained. The pAAV5 was cut with BssH II and MseI double digestion to recover the vector plasmid segment containing AAV-5 ITRs, then blunted with T4 DNA polymerase; pSNAV-GFP was cut with XhoI and BamHI enzyme to recover the CMV-PolyA segment, then blunted with T4 DNA polymerase after was recycled, and was then loaded in the vector plasmid segment containing AAV-5 ITRs. Then, the resistance gene neo^{r} from pSV2neo of Promega Co. was cut with Bgl II and Smal enzyme to recover the resistance gene neo^{r} followed by blunting with T4 DNA polymerase, and was then loaded in the vector plasmid segment containing AAV-5 ITRs. Accordingly, a recombinant plasmid pSNAV-N5 containing AAV-5 ITR elements was obtained.

A GFP gene-containing recombinant plasmid pSNAV-N5-GFP was constructed on the basis of the pSNAV-N5 plasmid, wherein the recombinant plasmid pSNAV-N5-GFP contained “ITR (AAV-5)-exogenous gene-ITR (AAV-5)” and a resistance gene neo^{r} (please see FIG. 13). The recombinant plasmid was introduced into BHK-21 cells (ATCC CCL-10) using Liposome Method, then was selectively cultured in G418 of 200 ug/ml for 10-15 days. Finally, the obtained resistance cell line was named as BHK/pSNAV-N5-GFP.

### EXAMPLE 4-4

### Preparation of rAAV/r2c5-GFP having the Serotype of AAV-5

BHK/pSNAV-GFP cells were infected with HSV1-r2c5. After cells exhibited pathology (36-72 h), the cell was repeatedly frozen and thawed for 4 times. Thus, the cell lysis solution contained rAAV/r2c5-GFP and helper viruses HSV1-r2c5. A low-speed centrifugation was carried out to remove the cell debris, and then the lysis solution was treated at 56° for 30 min so that the helper virus HSV1-r2c5 was deactivated. Accordingly, rAAV/r2c5-GFP having the serotype of AAV-5, which could be used to infect a cultured mammal cell in vitrolin vivo, was obtained from the supernatant of the cell lysis solution.

### EXAMPLE 4-5

### Transduction of Cultured Cells In Vitro by rAAV/r2c5-GFP

rAAV/r2c5-GFP viruses (MOI=1) were added to cultured BHK-21 cells (80% confluent). After 24-48 h, a great many green cells could be observed under a fluorescence microscope (excitation wavelength was 490 nm). That shows the resulting rAAV/r2c5-GFP virus is not only infectious but also capable of delivering an exogenous gene into a cell for expression.

### EXAMPLE 4-6

### Production of rAAV/r2c5-GFP viruses having the serotype of AAV-5 using Roller Bottle

pSNAV-GFPs were introduced into BHK-21 cells (bought from ATCC and cultured in RPMI1640 medium containing 10% FBS at 37°) using transfecting agent Lipofectamine (produced by GIBCO BRL Co), and then the cells were selectively cultured for 10-15 days in medium with G418 of 800 ug/ml. As a result, vector cells BHK/pSNAV-GFP for mixing cell clones were obtained. Then, after being enlarged propagation, the vector cells were cultured in four 35 cm²-square glass culture vessels; after confluent (about 8×10⁷ cells), the vector cells were digested with trypsin, inoculated into roller bottles (110×288 mm) followed by cultivation under a low-speed (1 rpm) rolling at 37°. The volume of the medium was 200 ml per roller bottle. 3 days later, the cells in the roller bottle were digested with trypsin and then introduced into 5 roller bottles for enlarged cultivation. After confluent (about 2×10⁹ cells), the medium was poured out and 5-10 ml of helper viruses HSV1-r2c5(MOI=0.5-2) was added in prior to the 1-2 hr of adsorption of the viruses at a low speed (1 rpm). Then, a serum-free 1640 medium (200 ml per roller bottle) was added in to culture the cells at 37° at a low speed (1 rpm). After pathology complete, cells could be shedding easily, the bottle tops were closed tightly before being violently vortexed until all the cells attached to the bottle side were eluted into the medium. After that, the cultures in the 5 roller bottles were pooled and gathered, and after the estimation of their volumes, they were divided into erlenmeyer flasks (specification: 500 ml), 250 ml per flask, for the next step—purification.

### EXAMPLE 4-7

### Purification of rAAV/r2c5-GFP Virus having the Serotype of AAV-5

Continuing the above Example. 25 ml of chloroform (10:1 v/v) was added to each erlenmeyer flask, which was placed in a shaker at 37° for 1˜1.5 hr under violent shaking before being taken out and sit at a room temperature for 10 min. DNase and RNase were then added in until the final concentration was 1 μg/ml. Then, after the mixture was blended gently until it became homogeneous, it was being digested at a room temperature for 30-60 min. Solid sodium chloride was added to a final concentration of 1 mol/L under shaking for dissolution. After that, the mixture was centrifugated at 12000 rpm at 4° for 15 min before the upper water phase was removed and the chloroform and deposits were discarded. Subsequently, PEG8000 was added in until the final concentration was 10% (w/v), and shaken to be dissolved. Then, the mixture was sit at 4° overnight before being centrifugated at 11000 rpm at 4° for 15 min. The supernatant was poured into a clean container (centrifuge tubes were turned upside down on the absorbent paper so that the supernatant could be drained to the full). After that, 5 ml PBS⁺ buffer was used to beat upon, elute and gather the deposits attached to the tube bottoms and tube sides of all the centrifuge tubes. Then the gathered deposits were aliquoted into plastic centrifuge tubes (specification: 1.5 ml), 0.6 ml per tube, before being extracted by chloroform in an equal volume. The extracted mixture were centrifugated at 12000 rpm at 4° for 5 min before the upper water phase was carefully aspirated via an antiseptic operation and then transferred into an antiseptic tube. The obtained liquid was just a condensed and purified rAAV/r2c5-GFP viral solution, of which the volume was 200 times condensed than its initial volume.

### EXAMPLE 4-8

### Electron Microscope Analysis of rAAV/r2c5-GFP Virus having the Serotype of AAV-5

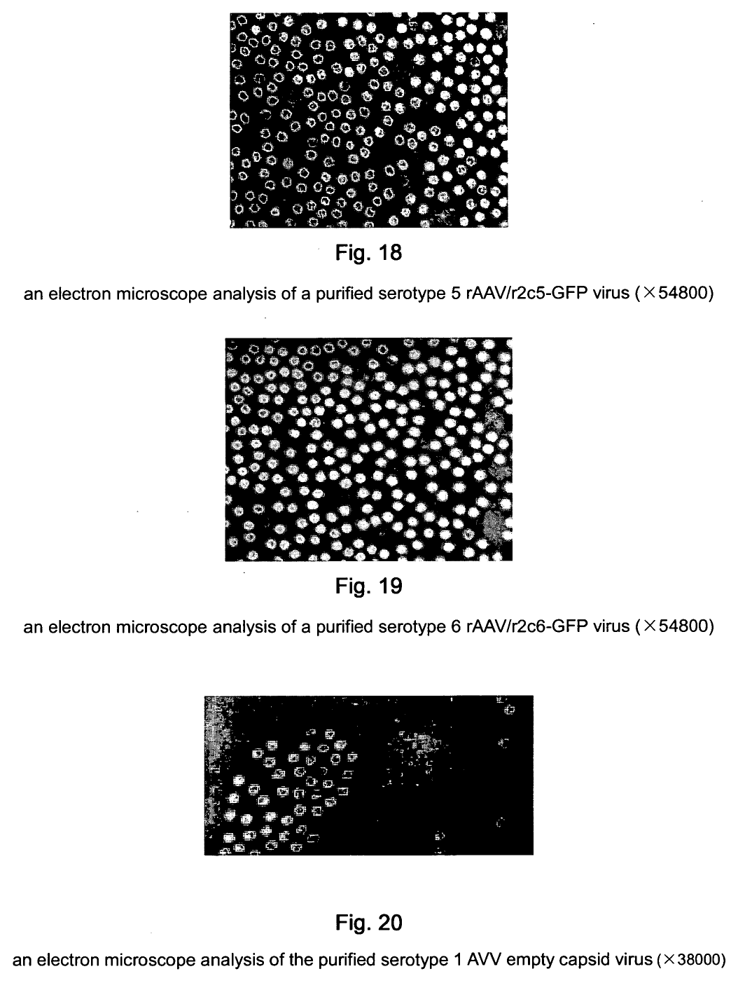

Solid viral particles with uniform and identical sizes (the particle sizes were around 20-24 mm) could be observed under an electron microscope after the purified rAAV/r2c5-GFP virus solution obtained in the above example was subjected to negative-staining. Please see FIG. 18 in the specification to see the detailed electron microscope results.

### EXAMPLE 4-9

### Titer Determination of rAAV/r2c5-GFP Virus having the Serotype of AAV-5

Continuing Example 4-7. The titer (particles/ml) of the rAAV/r2c5-GFP viruses in the purified viral solution was detected by Dot-Blotting Method using the Digoxigenin-Labeled (Boehringer Mannhein kit) GFP probe. 10 ul of the purified viral solution was diluted 1:10 with PBS²⁺ buffer. DNase and RNase were added in until the final concentration was 1 μg/ml. After the mixture was being digested at 37° for 1 hr and treated in boiling water-bath for 5 min, it was placed in ice bath and then diluted 1:10 with a dilution buffer before dot-blotting (1 ul/dot). Later, the membrane was baked at 120° C. for 30 min, the prehybridization was conducted at 68° C. for 1 hr followed by probe dot-blotting at 68° C. overnight. Afterwards, the membrane was washed and developed. Results: dots 1-4 were definitely positive while dot 5 was weak positive. Supposing the sensibility of detection of DNA by Dot-Blotting Method was 10⁶ molecules, it could be calculated that the viral titer=10⁴⁻⁵×10⁶×10×1000=10¹⁴⁻¹⁵ particles/ml.

### EXAMPLE 4-10

### Infectious Titer Determination of rAAV/r2c5-GFP Virus having the Serotype of AAV-5

At 37°, a 10% FBS-containing RPMI1640 medium and 5% CO₂ were used to culture HeLa cells, which were than seeded in a 24-well plate (5×10⁵ cell/well). After cultivation overnight, the medium was absorbed out; 10 ul of purified rAAV/r2c5-GFP viral solution was diluted 1:10 to 1 ml, then each well was added with 0.5 ml of viral solution with different dilutions and was cultured at 37° for 1 hr; subsequently, each well was added with 50 ul of Ad-5(MOI=5) and 0.5 ml of medium. After 36 hr of cultivation at 37°, green fluorescent cells could be observed under an inverted fluorescence microscope. After the number of green cells (represented by “n”, wherein 10<n<100) in a certain well had been counted, the viral titer of rAAV/r2c5-GFP could be calculated as follows: n×Times of Dilution×1000/5=n×10⁹×200=2n×10¹¹ TU/ml. Thus, it could be estimated that the infectious titer of the rAAV/r2c5-GFP virus was between 2×10¹² TU/ml and 2×10¹³ TU/ml.

### EXAMPLE 4-11

### Production and Purification of AVV Empty Capsid Viron

BHK-21 cells were cultured in roller bottles. After cells were confluent, helper viruses HSV1-r2c5 were added and pathological cell culture was obtained using the same process according to Example 4-6, and the AVV virus of the culture was extracted using the rAVV purification method according to the present invention. By observing the obtained viral solution under an electron microscope (Please see FIG. 23 in the specification), a great many viral particles with a high core density could be visualized. That showed the observed viral particles were empty capsids. The result shows that by using helper viruses HSV1-r2c5 to infect the BHK cell which does not be transfected by AVV vector DNA (ITR sequence not included), empty capsid of AVV viral can be produced effectively.

### EXAMPLE 5

### Method for Large-scale Production, Isolation, Purification of Recombinant Adeno-Associated Virus Serotype 6 Vectors and Uses Thereof

### EXAMPLE 5-1

### Construction of cos6-r2c6□UL2

AAV-1 was used as the template, and the corresponding cap6 (AAV-6) was amplified using PCR method (primers refer to Primer Sequences 9 and 10). The reaction conditions were 30 circulations of 94° for 30 sec, 55° for 30 sec and 72° for 3 min. A PCR segment of 2210 bp, cap6 was obtained. After double digesting with restrictive enzymes KpnI and XbaI, cap6 was linked with rep 2 of 1721 bp, which was digested from pSSV9 with KpnI and XbaI. The ligation product was inserted into XbaI site of plasmid pGEM-p3zf(+) (Promega Co.) to form p3zf-r2c6 plasmid. After digested from p3zf-r2c6 plasmid with XbaI, r2c6 (of around 4239 bp) was inserted into XbaI site of cos6. As a result, cos6-r2c6□UL2 was obtained.

Primer Sequence 9: Upstream primer of AAV-6 cap: 5′-TTTGCCGACAGGTACCAAAA-3′ (SEQ ID NO: 14);

Primer Sequence 10: Downstream primer of AAV-6 cap:

| | |
|---|---|
| 5′-TCTAGACACACAATTACAGGGGAC-3′. | (SEQ ID NO: 15) |

### EXAMPLE 5-2

### Preparation of Recombinant HSV1-r2c6

Cos6-r2c6□UL2 was mixed at equal molar ratio with five cosmids such as cos14, cos28, cos48, cos56 and the like. Then, the cos framework was removed by PacI enzyme (no need of remove the cos framework by isolation). After that, the mixture was extracted with phenol, phenol/chloroform (1:1) and chloroform, respectively. The supernatant was absorbed and collected, and DNA was precipitated using 2.5 times anhydrous ethanol. 20 ul of lipofactamine (GIBCO BRL) and 10 ul of DNA were used to co-transfect 80% confluent BHK-21 cells (about 2×10⁶ cells) according to the instructions, then homologous recombination of the 5 HSV-1 segments occurred inside the cells and accordingly HSV1-r2c6 recombinant viruses were generated, respectively. After 24 h of transfection, the cell was cultured in 1640 medium containing 2% FBS at 37°. The medium was changed every day. After 5 days, cells began to exhibit pathology. The supernatant of the medium was collected after complete pathology followed by centrifugating at 2000 r/min for 5 min. Then, the supernatant was aliquoted and stored at −20°. The obtained recombinant viruses were plaque-purified twice so that pure HSV1-r2c6 recombinant viruses were prepared.

### EXAMPLE 5-3

### Construction of AAV Packaging Cell Strain BHK/pSNAV-N6-GFP

AAV-6 and adenovirus 5 were used to infect 293 cells. 3 days later, the cells were frozen and thawed, and then were centrifugated (5800 g) for 30 min, and purified with CsCl as described in J.V.1997, 71: 8429-8436. The above-mentioned AAV-6 viruses were treated with 0.1% SDS and protease k of 0.2 mg/ml at 37° for 3 h, then was extracted with phenol/chloroform twice and extracted with chloroform once. Sodium acetate and alcohol were added to precipitate DNA. After deposition, the DNA was resuspensed by TE(PH8.0)at 95° for 5 min and then treated in 0.3-1.0 M NaCl at 50-60° for 2 h until the double-strands was annealed. Qiaex IIgel extraction kit (Qiagen) was used to purify the AAV-6 DNA band of about 5 kb running out from agarose gel, then the ends of the DNA band were flanked with Klenow large segment, and after addition of a linker of XbaI Linker (dCTCTAGAG) and purification, it was cut with XbaI, loaded in XbaI site of pGEM-3zf (a product of Promega Co.) and amplified in E. Coli DH5a Max Efficiency. After the single clones were picked out and the plasmid was extracted, those clones containing complete AAV-6 genomes were screened via restrictive enzyme and the rep2 probe method. Then, the plasmid was used to transfect BHK cells and 24 h later the cells were transfected by HSV-1. 2 days later, small molecular weight DNAs outside the cell chromosome were extracted using Hirt method, cut with DpnI enzyme, transferred by Southern blotting and hybridized using a rep probe, and the integrity of genomes was verified by Dimer band using a monomer. After all those, pAAV6 was obtained. The pAAV6 was cut with PmacI and BstEII double digestion to recover the vector plasmid segment containing AAV-6 ITRs, then blunted with T4 DNA polymerase; pSNAV-GFP was cut with XhoI and BamHI enzyme to recover the CMV-PolyA segment, then blunted with T4 DNA polymerase, and was then loaded in the vector plasmid segment containing AAV-6 ITRs. Then, the resistance gene neo^{r} was cut with Bgl II and Smal enzyme from pSV2neo of Promega Co., followed by blunted with T4 DNA polymerase, and was then loaded in the vector plasmid segment containing AAV-6 ITRs. Accordingly, a recombinant plasmid pSNAV-N6 containing AAV-6 ITR elements was obtained.

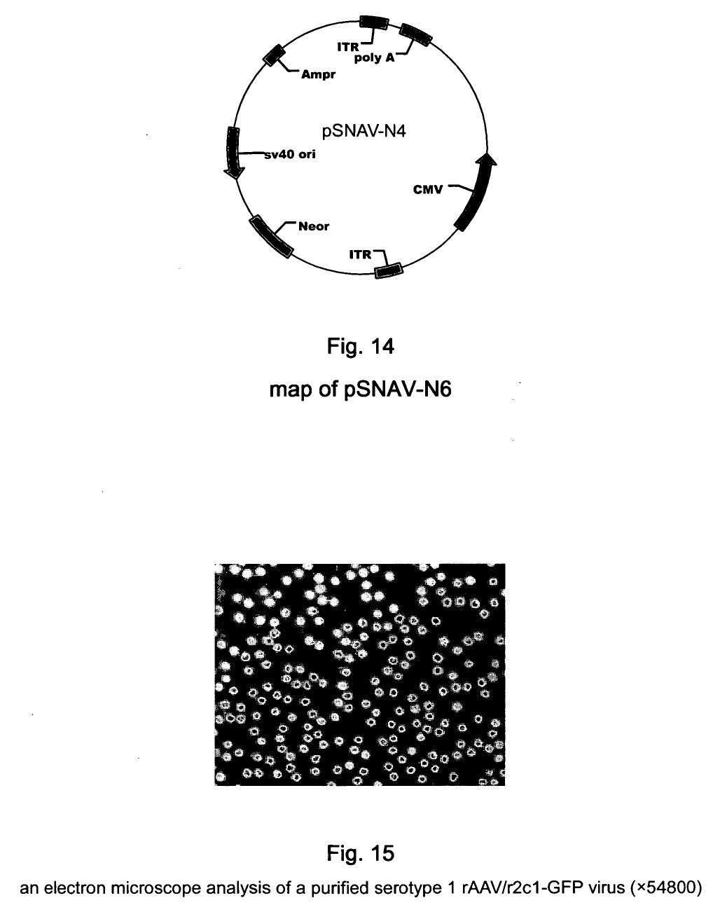

A GFP gene-containing recombinant plasmid pSNAV-N6-GFP was constructed on the basis of the pSNAV-N6 plasmid, wherein the recombinant plasmid pSNAV-N5-GFP contained “ITR (AAV-6)-exogenous gene-ITR (AAV-6)” and a resistance gene neo^{r} (please see FIG. 14). The recombinant plasmid was introduced into BHK-21 cells (ATCC CCL-10) using Liposome Method, and then was selectively cultured in G418 of 200 ug/ml for 10-15 days. Finally, the obtained resistance cell line was named as BHK/pSNAV-N6-GFP.

### EXAMPLE 5-4

### Preparation of rAAV/r2c6-GFP having the Serotype of AAV-6

BHK/pSNAV-GFP cells were infected with HSV1-r2c6. After cells exhibited pathology (36-72 h), the cell was repeatedly frozen and thawed for 4 times. Thus, the cell lysis solution contained rAAV/r2c6-GFP and helper viruses HSV1-r2c6. A low-speed centrifugation was carried out to remove the cell debris, and then the lysis solution was treated at 56° for 30 min so that the helper virus HSV1-r2c6 was deactivated. Accordingly, rAAV/r2c6-GFP having the serotype of AAV-6, which could be used to infect a cultured mammal cell in vitrolin vivo, was obtained from the supernatant of the cell lysis solution.

### EXAMPLE 5-5

### Transduction of Cultured Cells In Vitro by rAAV/r2c6-GFP

rAAV/r2c6-GFP viruses (MOI=1) were added to cultured BHK-21 cells (80% confluent). After 24-48 h, a great many green cells could be observed under a fluorescence microscope (excitation wavelength was 490 nm). That shows the resulting rAAV/r2c6-GFP virus is not only infectious but also capable of delivering an exogenous gene into a cell for expression.

### EXAMPLE 5-6

### Production of rAAV/r2c6-GFP Viruses having the Serotype of AAV-6 Using Roller Bottle

pSNAV-GFPs were introduced into BHK-21 cells (bought from ATCC and cultured in RPMI1640 medium containing 10% FBS at 37°) using transfecting agent Lipofectamine (produced by GIBCO BRL Co), and then the cells were selectively cultured for 10-15 days in medium with G418 of 800 ug/ml. As a result, vector cells BHK/pSNAV-GFP for mixing cell clones were obtained. Then, after being enlarged propagation, the vector cells were cultured in four 35 cm²-square glass culture vessels; after confluent (about 8×10⁷ cells), the vector cells were digested with trypsin, inoculated into roller bottles (110×288 mm) followed by cultivation under a low-speed (1 rpm) rolling at 37°. The volume of the medium was 200 ml per roller bottle. 3 days later, the cells in the roller bottle were digested with trypsin and then introduced into 5 roller bottles for enlarged cultivation. After confluent (about 2×10⁹ cells), the medium was poured out and 5-10 ml of helper viruses HSV1-r2c6(MOI=0.5-2) was added in prior to the 1-2 hr of adsorption of the viruses at a low speed (1 rpm). Then, a serum-free 1640 medium (200 ml per roller bottle) was added in to culture the cells at 37° at a low speed (1 rpm). After pathology complete, cells could be shedding easily, the bottle tops were closed tightly before being violently vortexed until all the cells attached to the bottle side were eluted into the medium. After that, the cultures in the 5 roller bottles were collected and gathered, and after the estimation of their volumes, they were divided into erlenmeyer flasks (specification: 500 ml), 250 ml per flask, for the next purification step.

### EXAMPLE 5-7

### Purification of rAAV/r2c5-GFP virus having the serotype of AAV-6

Continuing the above Example. 25 ml of chloroform (10:1 v/v) was added to each erlenmeyer flask, which was placed in a shaker at 37° for 1˜1.5 hr under violent shaking before being taken out and sit at a room temperature for 10 min. DNase and RNase were then added in until the final concentration was 1 μg/ml. Then, after the mixture was blended gently until it became homogeneous, it was being digested at a room temperature for 30-60 min. Solid sodium chloride was added to a final concentration was 1 mol/L under shaking for dissolution. After that, the mixture was centrifugated at 12000 rpm at 4° or 15 min before the upper water phase was removed and the chloroform and deposits were discarded. Subsequently, PEG8000 was added in until the final concentration was 10% (w/v), and shaken to be dissolved. Then, the mixture was sit at 4° overnight before being centrifugated at 11000 rpm at 4° for 15 min. The supernatant was poured into a clean container (centrifuge tubes were turned upside down on the absorbent paper so that the supernatant could be drained to the full). After that, 5 ml PBS⁺ buffer was used to beat upon, elute and gather the deposits attached to the tube bottoms and tube sides of all the centrifuge tubes. Then the gathered deposits were divided into plastic centrifuge tubes (specification: 1.5 ml), 0.6 ml per tube, before being extracted by chloroform in an equal volume. The extracted mixture were centrifugated at 12000 rpm at 4° for 5 min before the upper water phase was carefully aspirated via an antiseptic operation and then transferred into an antiseptic tube. The obtained liquid was just a condensed and purified rAAV/r2c6-GFP viral solution, of which the volume was 200 times condensed than its initial volume.

### EXAMPLE 5-8

### Electron Microscope Analysis of rAAV/r2c6-GFP Virus having the Serotype of AAV-6

Solid viral particles with uniform and identical sizes (the particle sizes were around 20-24 mm) could be observed under an electron microscope after the purified rAAV/r2c6-GFP virus solution obtained in the above example was subjected to negative-staining. Please see FIG. 19 in the specification to see the detailed electron microscope results.

### EXAMPLE 5-9

### Titer Determination of rAAV/r2c6-GFP Virus having the Serotype of AAV-6

Continuing Example 5-7. The titer (particles/ml) of the rAAV/r2c6-GFP viruses in the purified viral solution was detected by Dot-Blotting Method using the Digoxigenin-Labeled (Boehringer Mannhein kit) GFP probe. 10 ul of the purified viral solution was diluted 1:10 with PBS²⁺ buffer. DNase and RNase were added in until the final concentration was 1 μg/ml. After the mixture was being digested at 37° for 1 hr and treated in boiling water-bath for 5 min, it was placed in ice bath and then diluted 1:10 with a dilution buffer before dot-blotting (1 ul/dot). Later, the membrane was baked at 120° C. for 30 min, the prehybridization was conducted at 68° C. for 1 hr followed by probe dot-blotting at 68° C. overnight. Afterwards, the membrane was washed and developed. Results: dots 1-4 were definitely positive while dot 5 was weak positive. Supposing the sensibility of detection of DNA by Dot-Blotting Method was 10⁶ molecules, it could be calculated that the viral titer=10⁴⁻⁵=10⁶10×1000=10¹⁴⁻¹⁵ particles/ml.

### EXAMPLE 5-10

### Infectious Titer Determination of rAAV/r2c6-GFP Virus having the Serotype of AAV-6

At 37°, a 10% FBS-containing RPMI1640 medium and 5% CO₂ were used to culture HeLa cells, which were than seeded in a 24-well plate (5×10⁵ cell/well). After cultivation overnight, the medium was absorbed out; 10 ul of purified rAAV/r2c6-GFP viral solution was diluted 1:10 to 1 ml, then each well was added with 0.5 ml of viral solution with different dilutions and was cultured at 37° for 1 hr; subsequently, each well was added with 50 ul of Ad-5(MOI=5) and 0.5 ml of medium. After 36 hr of cultivation at 37°, green fluorescent cells could be observed under an inverted fluorescence microscope. After the number of green cells (represented by “n”, wherein 10<n<100) in a certain well had been counted, the viral titer of rAAV/r2c6-GFP could be calculated as follows: n×Times of Dilution×1000/5=n×10⁹200=2n×10¹¹ TU/ml. Thus, it could be estimated that the infectious titer of the rAAV/r2c6-GFP virus was between 2×10¹² TU/ml and 2×10¹³ TU/ml.

### EXAMPLE 5-11

### Production and Purification of AVV Empty Capsid Virion

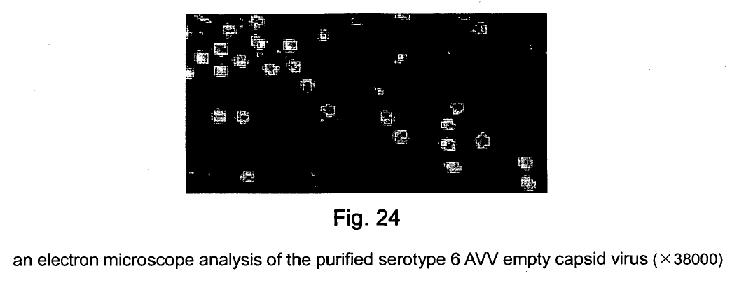

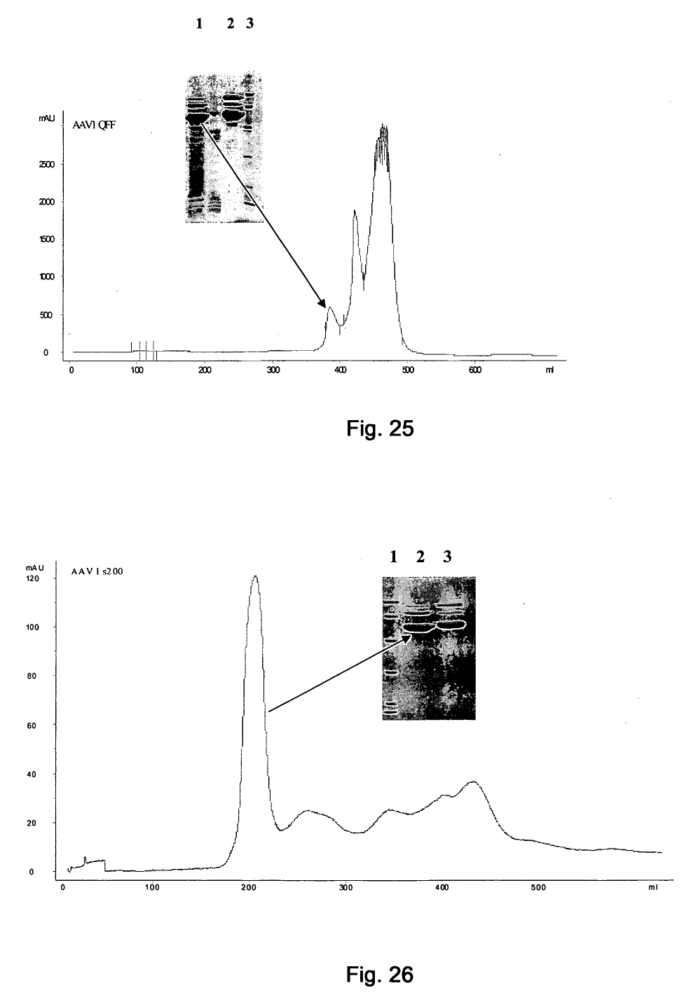

BHK-21 cells were cultured in roller bottles. After cells were confluent, helper viruses HSV1-r2c6 were added and pathological cell culture was obtained using the same process according to Example 5-6, and the AVV virus of the culture was extracted using the rAVV purification method according to the present invention. By observing the obtained viral solution under an electron microscope (Please see FIG. 24 in the specification), a great many viral particles with a high core density could be visualized. That showed the observed viral particles were empty capsids. The result shows that by using helper viruses HSV1-r2c6 to infect the BHK cell which does not be transfected by AVV vector DNA (ITR sequence not included), empty capsid of AVV viral can be produced effectively.

1. A recombinant herpes simplex virus, comprising a heterologous nucleotide sequence selected from the group consisting of the sequences represented by SEQ ID NO: 1, SEQ ID NO: 2, SEQ ID NO: 3, SEQ ID NO: 4, and SEQ ID NO: 5.

2. The recombinant herpes simplex virus according to claim 1, wherein the heterologous nucleotide sequence is inserted at the XbaI site of the UL2 gene or the UL44 gene of the HSV genome.

3. The recombinant herpes simplex virus according to claim 1, wherein the heterologous sequence represented by SEQ ID NO: 1, SEQ ID NO: 2, SEQ ID NO: 3, SEQ ID NO: 4, or SEQ ID NO: 5 is inserted in a nonessential gene region of the HSV genome.

4. A method for the production of a recombinant herpes simplex virus, comprising obtaining at least one DNA segment represented by a sequence selected from the group consisting of SEQ ID NO: 1, SEQ ID NO: 2, SEQ ID NO: 3, SEQ ID NO: 4, and SEQ ID NO: 5; and inserting the DNA segment into an HSV genome using gene engineering method(s), thereby obtaining the recombinant herpes simplex virus.

5. (canceled)

6. A method for large-scale production and preparation of a recombinant adeno-associated virus serotype 1, 3, 4, 5, or 6, the method comprising:
(1) obtaining a recombinant herpes simplex virus comprising a heterologous nucleotide sequence selected from the group consisting of the sequences represented by SEQ ID NO: 1, SEQ ID NO: 2, SEQ ID NO: 3, SEQ ID NO: 4, and SEQ ID NO: 5;
(2) establishing “a vector cell”, namely, a recombinant AAV vector cell strain;
(3) infecting the vector cell strains with the recombinant herpes simplex virus; and
(4) culturing the infected vector cell strain under conditions whereby recombinant adeno-associated viruses are produced.

7. A method for isolation and purification of recombinant adeno-associated virus serotype comprising:
1) adding chloroform to a crude lysis solution comprising recombinant adeno-associated virus-containing cells to deactivate any HSV helper viruses, lyse cells, and denature and precipitate a great many cell proteins to obtain cell lysis solution;
2) adding solid sodium chloride to the cell lysis solution until the final concentration is 1.0 to 1.2 mol/L with stirring for dissolution, then centrifugating the mixture and leaving the supernatant;
3) precipitating rAAV with PEG/NaCl, adding solid polyethylene glycol to the sodium chloride-containing supernatant of step 2) with stirring for dissolution, letting the mixture sit, then centrifugating the mixture and discarding the supernatant but leaving the precipitate;
4) treating the cell lysis solution with DNaseI and RNase to degrade the nucleic acid, dissolving the precipitate of step 3), and adding DNaseI and RNase to dissolve the residual nucleic acid apart from the rAAV viral particles;
5) using chloroform to extract and remove other proteins and the residual PEG, adding chloroform to extract, and then, centrifugating the mixture and removing the upper water phase;
6) removing salts via dialysis; and
7) further purifying rAAV via density gradient centrifugation or affinity chromatography.

8. A recombinant vector plasmid pSNAV-NX, said plasmid comprising an ITR at each of the two ends of an AAV genome selected from the group consisting of the AAV-1, AAV-3, AAV-4, AAV-5 and AAV-6 genomes; and further comprising an immediate early enhancer and a promoter of cytomegalovirus, a multiple cloning site and a polyA signal successively intervening between the two ITRs, and a neomycin resistant gene expression cassette flanking the outside of at least one of the ITRs.

9. The method of claim 7 wherein the recombinant adeno-associated virus has a serotype selected from the group consisting of serotypes 1, 3, 4, 5, and 6.

10. A method for purification of recombinant adeno-associated virus serotype 1, 3, 4, 5, or 6, the method comprising: obtaining a solution containing rAAV of serotype 1, 3, 4, 5, or 6; adjusting the conductance value of obtained rAAV solution before passing through an ion exchange column which has been balanced by a buffer, balancing the ion exchange column using a buffer again; eluting the ion exchange column with a salt-containing buffer and collecting the elution peaks; passing the collected elution peaks through a molecular sieve column which has been balanced by a buffer; and washing the column with a buffer again.

11. The method of claim 7 wherein the rAAV is an empty capsid of an AAV serotype selected from the group consisting of serotypes 1, 3, 4, 5, and 6.
